(19) United States
(12) Patent Application Publication
(10) Pub. No.: US 2007/0016014 A1
(43) Pub. Date: Jan. 18, 2007

(54) Radio therapy apparatus and operating method of the same

(76) Inventors: Kenji Hara, Hiroshima (JP); Makoto Akatsu, Hiroshima (JP); Noriyuki Kawata, Hiroshima (JP)

Correspondence Address: WENDEROTH, LIND & PONACK, L.L.P., 2033 K STREET N. W., SUITE 800, WASHINGTON, DC, 20006-1021, US

(21) Appl. No.: 11/152,504

(22) Filed: Jun. 15, 2005

(51) Int. Cl. A61B 5/05; A61N 5/10

(52) U.S. Cl. 600/426; 378/65

(57) ABSTRACT

A radio-therapy apparatus includes a radiation head configured to irradiate a therapeutic radiation, and an image processing section configured to generate an image of a diseased portion of a specimen from a result of detection of the diseased portion while tracking the diseased portion to which the therapeutic radiation is irradiated from the radiation head. A control section controls the radiation head and the image processing section such that a period containing the generation of the image and the irradiation of the therapeutic radiation is repeated and the detection of the diseased portion in a next period is started prior to an end of a current period. A recording section records the image of the diseased portion generated by the image processing section in order.

### CROSS REFERENCE

This application is related to the U.S. patent application Ser. No. 11/067013. The disclosure of the U.S. patent application Ser. No. 11/067013 is incorporated herein by reference.

### BACKGROUND OF THE INVENTION

1. Field of the Invention

The present invention relates to a radio-therapy apparatus and a method of operating a radio-therapy apparatus, and more particularly relates to a technique for recording a therapy history at a time of radio-therapy.

2. Description of the Related Art

Conventionally, a radio-therapy apparatus for treating a cancer and a tumor by using radiation is known. A radio surgery therapy apparatus, a linear accelerator therapy apparatus and the like are known as a three-dimensional radio-therapy apparatus for stereo-taxic multi-orbit radiation. The stereo-taxic multi-orbit radiation is a radio-therapy method of intensively irradiating a radiation beam to a small nidus from many directions to improve the radio-therapy effect, and also to suppress an exposure amount of peripheral tissues to a minimum.

The conventional radio surgery therapy apparatus intensively irradiates a thin radiation beam from a radiation irradiating unit fixed to the therapy apparatus, to a particular small region at a high precision. A gamma-ray source or a linear acceleration unit is used as the radiation irradiating unit. In the radio surgery therapy apparatus, a fixing tool (to be referred to as a frame) is used for precisely positioning a diseased portion. Thus, the diseased portion such as a skull and the like of a patient or the peripheral portion thereof is mechanically fixed. This frame is used as a coordinate reference tool for positioning, and diagnosis images are taken by apparatuses such as X-ray CT (Computed Tomography), MRI, DAS (Digital Subtraction Angiography) to determine the accurate position and shape of the diseased portion. Then, while this frame is kept, the patient is mechanically fixed to the radio-therapy apparatus provided with the radiation irradiating unit and a collimating mechanism for collimating the radiation beam irradiated from the irradiating unit and concentrating the radiation beam to a small region. Consequently, a radiation field is adjusted mechanically precisely to the small region, and the precise stereo-taxic radiation is carried out. If the diseased portion is spherical, a necessary radiation dose amount can be attained only one radiation. If the diseased portion is non-spherical, the positioning is repeated several times in accordance with the shape of the radiation field. Simultaneously, the aperture of a collimator is selected each time, and the radio-therapy is carried out.

The radio surgery therapy apparatus has a simple structure, the operation procedure is very simple, and the high reliability is obtained. In addition, if the radiation field does not move with respect to the skull like a head, the extremely precise positioning and radiation are possible. However, since the radiation field of the radiation irradiating unit is fixed, the stereo-taxic radio-therapy cannot be carried out on the body that the radiation field such as the tumor is moved by the influence of the motions and situations of the organs below a neck, e.g., the respiration and beat, the peristalsis, a urine amount inside a bladder, and the like. Also, strictly speaking, the radiation beam is not always irradiated while the diseased portion is observed in real time.

Also, in the conventional linear acceleration therapy apparatus, a large gantry is rotated by 360 degrees around one shaft parallel to its installation surface, and an isocentric radio-therapy is consequently carried out. Moreover, the radiation from various directions is possible by 2-dimensionally moving a therapeutic bed in up and down directions or in horizontal direction, and by inclining on a horizontal plane. Also, the conventional linear acceleration treating apparatus copes with the radiation field of a complex shape by using MLC (Multi Leaf Collimator), and is possible to control a radiation dose amount distribution to carry out a precise radio-therapy (IMRT: Intensity Modulated Radio Therapy).

However, in this linear acceleration therapy apparatus, it is impossible to carry out a position control at a high speed. Thus, it is impossible to track a radiation field moving at the high speed due to the beat of the heart and the like in real time. As a monitoring method of the radiation field, the therapeutic X-ray is used for a lineac graphic. However, since the therapeutic X-ray has a strong transmission and many scattered lines, the therapeutic X-ray is unsuitable for the real time monitor of the radiation field.

With only a respiratory motion, synchronous radiation is carried out by using a respiratory synchronizing apparatus. In this method, the position of the diseased portion is predicted by using a predetermined method, because the image of the diseased portion cannot be imaged in real time. When the diseased portion is predicted to arrive at a predetermined radiation field, an irradiating apparatus is triggered to irradiate the therapeutic radiation beam. In the predicting method, the movement of the diseased portion is predicted by optically tracking a marker attached to the diseased portion or directly measuring a flow rate of exhalation. Thus, the state of the respiration of the patient is predicted. However, since the synchronous radiation is carried out by predicting the position of the diseased portion and irradiating the radiation beam towards the predicted position, the therapeutic radiation is not necessarily carried out the while tracking the diseased portion in the real time.

Also, as other three-dimensional radio-therapy apparatuses, there are known an apparatus for isocentrically driving an electronic linear accelerator and an apparatus for driving the electronic linear accelerator along a gantry of a predetermined shape.

As the apparatus for isocentrically driving the electronic linear accelerator, there is known an apparatus provided with a small electronic linear accelerator at a tip of a general robot arm for industry. The accurate shape and position of the diseased portion is determined by using an X-ray CT, an MRI and the like, in relation to a marker of a small gold plate or the like, which is embedded in the vicinity of land marking body tissue and the diseased portion such as a skull and a chest. At the time of the therapeutic radiation, while the movement of the land mark is monitored by two X-ray cameras having different sight lines, the radiation field is corrected, so that the precise radiation can be carried out. This apparatus can essentially carry out a non-isocentric radio-therapy based on a movable performance of a 6-free degree robot arm.

However, in this apparatus, although a fixing tool is used for fixing the head in case of the head therapy, the irradiation is not always irradiated while the images of the diseased portion are directly observed. That is, the imaging by the X-ray camera is not carried out during the irradiation of the radiation beam. The imaging is completed prior to start of the irradiation, and the irradiation is started after the radiation field is checked. Thus, in this case, the irradiation field is not monitored in the real time, too. Also, the electronic linear accelerator is heavy in weight. Therefore, in order to apply the precise tracking radiation to the radiation filed under the quick motion such as the beat in real time while the electronic linear accelerator is held on the tip of the robot arm of a cantilever structure, it is necessary to solve a problem of inertia. Also, the industrial robot arm does not insure the absolute precision to a specified space coordinate, and it only insures the repetition precision through teaching. For this reason, the teaching and a work related to it are required prior to the actual therapeutic radiation.

An apparatus for driving the electronic linear accelerator along a gantry of a predetermined shape is disclosed in, for example, Japanese Laid Open Patent Application (JP-A-Heisei 8-504347 corresponding to International Application Number: PCT/US93/11872: a first conventional example) and Japanese Laid Open Patent Application (JP-A-Heisei 6-502330 corresponding to International Application Number: PCT/US91/07696: a second conventional example). The stereo-taxic radio-therapy apparatus in this technique includes a C-arm type X-ray camera having two rotation shafts and a curing electronic linear accelerator having two rotation shafts. As compared with the conventional electronic linear accelerator that can be only rotated around one shaft, the other rotation shaft is added to permit three-dimensional radiation. However, an irradiating method is isocentric and is similar to the radio surgery therapy apparatus in the point that the head needs to be fixed by the frame. Also, this conventional example is different from the radio surgery therapy apparatus in that the large gantry is driven with the two shafts.

The diseased portion of the patient is moving even during the radio-therapy. In particular, below the neck, the radiation field such as the tumor always moves due to the influence of the motions and situations of organs such as the respiration and the beat, the peristalsis, and a urine amount inside a bladder. For example, even when the patient lies, the body becomes gradually flat. Also, although the respiration and beat are the cyclic operations, the motions of the respective organs associated with it do not always pass through the same route every time. On the other hand, the beat that is one of the rapidest motions is 1 or two times/second. Therefore, if the motion of the radiation field is tried to be acquired accurately in real time, it is said that the image imaging technique of about 30 images per second is required. If trying to track the radiation field accurately in real time and to irradiate the radiation, it is necessary to accurately orient the radiation irradiating head towards the radiation field for each 1/30 seconds.

By the way, when the foregoing radio-therapy apparatus is used for the radio-therapy, the conservation of a therapy history is obligated. In order to cope with this obligation in the therapy using the conventional radio-therapy apparatus, a transmission image of the radiation field is imaged and recorded immediately after the radio-therapy to check the radiation position of the therapeutic radiation. This recorded transmission image is filed to the authorities in response to a request, and also provided to a doctor. However, since the radiation record during the radio-therapy is not always recorded, the portion to which the radio-therapy is actually carried out is unclear. Thus, it cannot be said to be sufficient as a record of the radio-therapy history, which brings about a problem that a new radio-therapy plan is difficult. For this reason, it is desired to clarify the portion to which the therapeutic radiation has been irradiated during the radio-therapy.

Also, in the radio-therapy using the conventional radio-therapy apparatus, the radiation beam for a planned dose amount is irradiated from a preliminarily planned direction, and a total radiation dose amount is recorded on a clinic card or the like. Also, the transmission image of the radiation field is imaged immediately after the radio-therapy and recorded as the radiation field. Then, this recorded transmission image is provided to the doctor and the Ministry of Health, Labor and Welfare in response to the request. Thus, since there is no data indicating the radiation state under the radio-therapy, the portion to which the therapeutic radiation is irradiated and the radiation dose amount at that time are unobvious. Therefore, there is a problem that it is difficult for the doctor to check the therapy state, to plan a next therapy action and to determine whether or not a present radio-therapy is proper. For these reasons, the method is demanded in which the doctor can easily check the therapy status, determine whether or not the present radio-therapy is proper, and plan the next therapy action.

Also, in the therapy using the conventional radio-therapy apparatus, the situation inside a therapy room is monitored with a television camera, and the radiation dose amount is displayed. If there is any trouble, an action that the doctor stops the therapy is carried out. However, whether or not the therapy content is proper cannot be determined, and the quality of the therapy is not evident. Therefore, the development of the method is desired that can determine whether or not the therapy is proper, by using the total data including the status of the therapy portion.

Moreover, in the therapy using the conventional radio-therapy apparatus, the therapeutic radiation dose amount is measured by using a radiation dose amount indicator mounted in a radiation head. This is recorded together with a head position space coordinate of the radiation head. However, this method has a problem that the coordinate data of the diseased portion of the patient is not known, and the direction of the radiation is unclear, and the check for the therapy plan is difficult.

### SUMMARY OF THE INVENTION

Therefore, an object of the present invention is to provide a radio-therapy apparatus, in which a therapy plan can be easily planned after a radio-therapy is carried out on a specimen, and an operating method of the radio-therapy apparatus.

In an aspect of the present invention, a radio-therapy apparatus includes a radiation head configured to irradiate a therapeutic radiation, and an image processing section configured to generate an image of a diseased portion of a specimen from a result of detection of the diseased portion while tracking the diseased portion to which the therapeutic radiation is irradiated from the radiation head. A control section controls the radiation head and the image processing section such that a period containing the generation of the image and the irradiation of the therapeutic radiation is repeated and the detection of the diseased portion in a next period is started prior to an end of a current period. A recording section records the image of the diseased portion generated by the image processing section in order.

Here, the radio-therapy apparatus may further include a calculating section configured to calculate a therapy record by the therapeutic radiation based on an operation state of the radiation head. The therapy record may include a therapeutic dose amount of the therapeutic radiation and an estimated absorption dose amount estimated to be absorbed by the specimen, and the recording section may record the therapy record calculated by the calculating section together with the image of the diseased portion in order.

Also, the calculating section may calculate the therapy record every irradiation direction of the therapeutic radiation.

Also, the radio-therapy apparatus may further include a display configured to display the image of the diseased portion and the therapy record which are recorded in the recording section. In this case, the display may display a moment value of the therapeutic radiation and an accumulation value of the therapeutic radiation. In this case, the display may display a data indicating whether a radiation field of the therapeutic radiation is proper.

Also, the recording section may record a data indicating a position of the diseased portion to the radiation head in addition to the image.

Also, the control section may control the radiation head and the image processing section such that the irradiation of the therapeutic radiation in the current period is carried out after the detection of the diseased portion.

In another aspect of the present invention, an operating method of a radio-therapy apparatus is achieved by irradiating a therapeutic radiation; by generating an image of a diseased portion of a specimen from a result of detection of the diseased portion while tracking the diseased portion of a specimen to which therapeutic radiation is irradiated from a radiation head; by controlling the irradiating and the generating such that a period containing the generating the image and the irradiating the therapeutic radiation is repeated and the detection of the diseased portion in a next period is started prior to an end of a current period; and by recording the image of the diseased portion in order.

Here, the operating method of the radio-therapy apparatus may be achieved by further calculating a therapy record by the therapeutic radiation based on an operation state of the radiation head. The therapy record may include a therapeutic dose amount of the therapeutic radiation and an estimated absorption dose amount estimated to be absorbed by the specimen. The recording may be achieved by recording the therapy record together with the image of the diseased portion in order.

Also, the calculating may be achieved by calculating the therapy record every irradiation direction of the therapeutic radiation.

Also, the operating method of the radio-therapy apparatus may be achieved by further displaying the image of the diseased portion and the therapy record. The displaying may be achieved by displaying a moment value of the irradiation dose amount of the therapeutic radiation and an accumulation value thereof. In this case, the displaying may be achieved by displaying a data indicating whether a radiation field of the therapeutic radiation is proper.

Also, the recording may be achieved by recording a data indicating a position of the diseased portion to the radiation head in addition to the image.

Also, the controlling may be achieved by controlling the irradiating and the generating such that the irradiation of the therapeutic radiation in the current period is carried out after the detection of the diseased portion.

### BRIEF DESCRIPTION OF THE DRAWINGS

FIG. 1 is a side view showing a configuration of a radio-therapy apparatus according to an embodiment of the present invention;

FIG. 2 is a front view showing the configuration of the radio-therapy apparatus according to the embodiment of the present invention;

FIG. 3 is a perspective view showing the configuration of the radio-therapy apparatus according to the embodiment of the present invention;

FIG. 4 is a perspective view showing another configuration of the radio-therapy apparatus according to the embodiment of the present invention;

FIG. 5 is a block diagram showing a configuration of a control system of the radio-therapy apparatus according to the embodiment of the present invention;

FIG. 6A is a front view showing a position calibration of the radio-therapy apparatus according to the embodiment of the present invention;

FIG. 6B is a side view showing the position calibration of the radio-therapy apparatus according to the embodiment of the present invention;

FIG. 7A is timing charts showing a timing of the operation of processing a diagnosis image in the radio-therapy apparatus according to the embodiment of the present invention;

FIG. 7B is timing charts showing a timing of the image tracking calculation based on the diagnosis image after the processing and a swinging operation of an X-ray head;

FIG. 7C is a timing chart showing a timing of therapeutic radiation from a therapeutic X-ray head;

FIG. 8 is a perspective view showing an irradiation situation of the therapeutic radiation through the X-ray head of the radio-therapy apparatus according to the embodiment of the present invention;

FIG. 9 is a sectional view along an A-A′ line in FIG. 8 to show a manner of irradiation of the therapeutic X-ray while swinging the X-ray head of the radio-therapy apparatus according to the embodiment of the present invention;

FIG. 10 is a sectional view along a B-B′ line in FIG. 8 to show a manner of irradiation of the therapeutic X-ray while swinging the X-ray head of the radio-therapy apparatus according to the embodiment of the present invention;

FIGS. 11A to 11F are a flow chart showing a procedure of a pseudo non-isocentric therapy in the radio-therapy apparatus according to the embodiment of the present invention;

FIG. 12A is a view showing a relation between a diseased portion and a definition region in the radio-therapy apparatus according to the embodiment of the present invention;

FIGS. 12B to 12E are diagrams showing a relation between the diseased portion and a boarder line;

FIG. 13 is a graph showing an example of a brightness distribution in a diagnosis image in the radio-therapy apparatus according to the embodiment of the present invention;

FIG. 14 is a diagram showing an example of tracking the diseased portion in the radio-therapy apparatus according to the embodiment of the present invention;

FIG. 15 is a diagram showing a display example onto a display in the radio-therapy apparatus according to the embodiment of the present invention;

FIG. 16 is a view showing another display example onto the display in the radio-therapy apparatus according to the embodiment of the present invention;

FIGS. 17A and 17B are diagrams showing an radiation direction of the therapeutic radiation and the dose amount in the radio-therapy apparatus according to the embodiment of the present invention;

FIG. 18 is a side view showing a configuration of a first modification example of the radio-therapy apparatus according to the embodiment of the present invention;

FIG. 19 is a front view showing a configuration of a rotating drum (therapy gantry) in a second modification example of the radio-therapy apparatus according to the embodiment of the present invention;

FIG. 20 is a front view showing a configuration of a rotating drum (therapy gantry) in the second modification example of the radio-therapy apparatus according to the embodiment of the present invention; and

FIG. 21 is a front view showing a configuration of a third modification example of the radio-therapy apparatus according to the embodiment of the present invention.

### DESCRIPTION OF THE PREFERRED EMBODIMENTS

Hereinafter, a radio-therapy apparatus of the present invention will be described in detail with reference to the attached drawings.

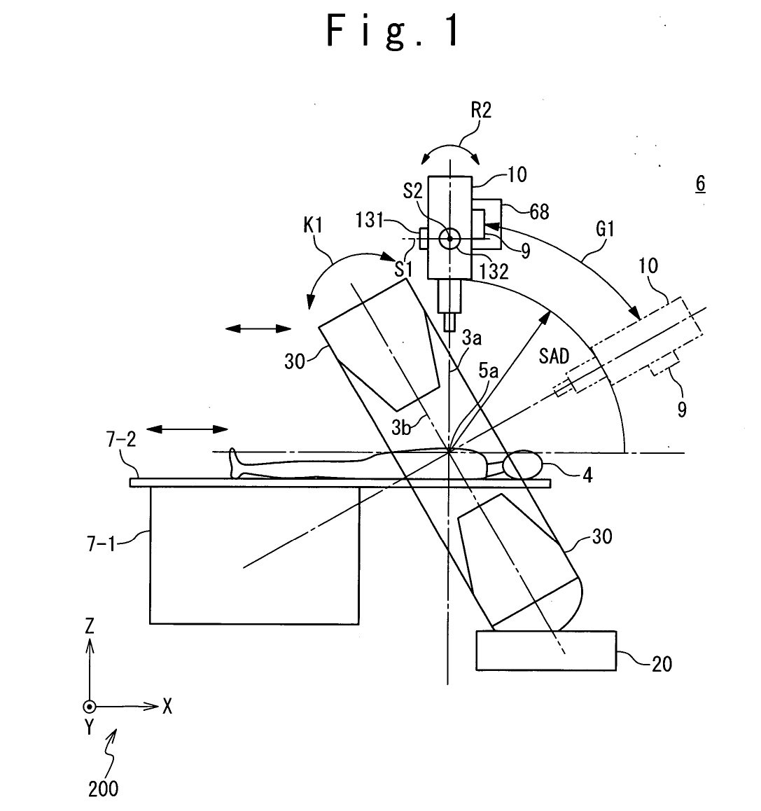

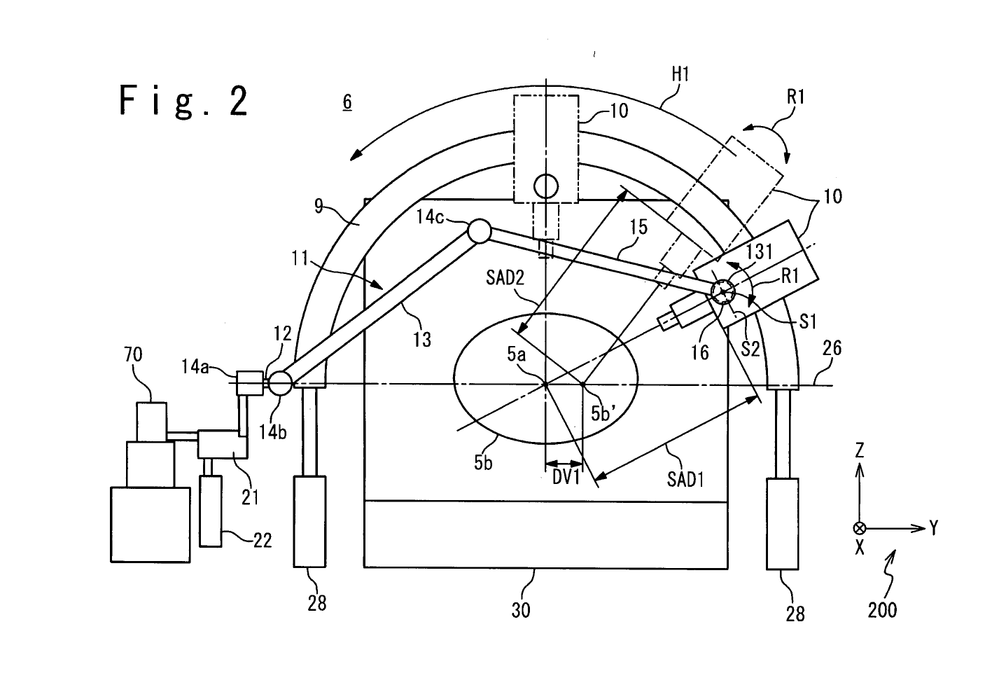

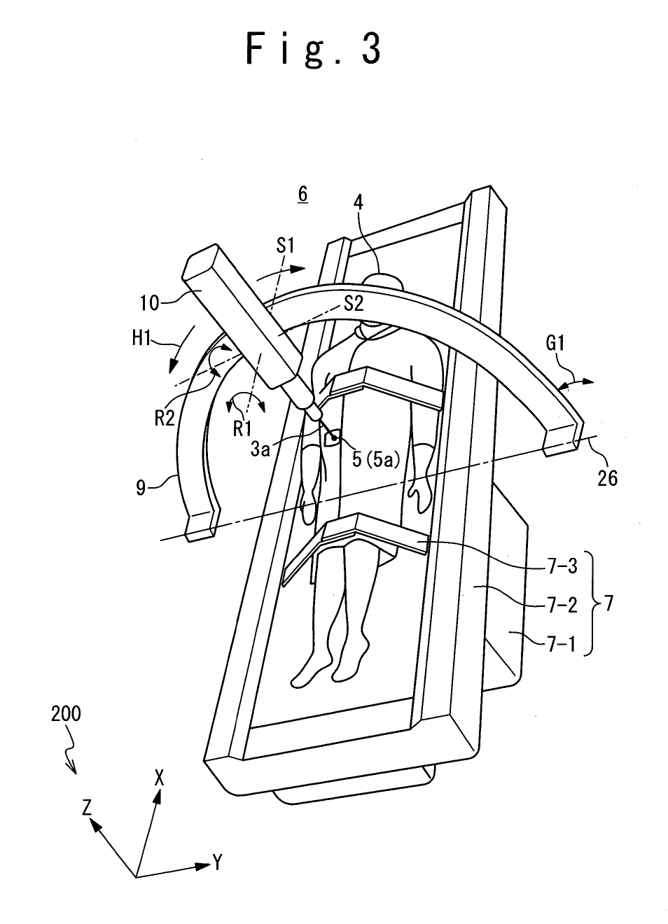

FIG. 1 is a side view of the radio-therapy apparatus according to an embodiment of the present invention, FIG. 2 is its front view, and FIG. 3 is a perspective view. It should be noted that a part of the radio-therapy apparatus is omitted in these drawings. A coordinate system 200 indicates a three-dimensional orthogonal coordinate system having an X-axis, a Y-axis and a Z-axis in FIGS. 1 to 3.

A radio-therapy apparatus 6 includes a therapeutic bed system 7, an X-ray head 10, a first swinging mechanism 131, a second swinging mechanism 132, an arc guide rail 9, a microwave generating unit 70, a tracking type waveguide system 11 and a real time imager 30.

The therapeutic bed system 7 is provided with a bed driving system 7-1, a therapeutic bed 7-2 and a patient fixing unit 7-3. The therapeutic bed 7-2 movably supports a patient 4 targeted for the radio-therapy and mounted on an X-Y table on the bed driving system 7-1. The patient fixing unit 7-3 fixes the patient 4 onto the therapeutic bed 7-2. The bed driving system 7-1 uses its built-in driving mechanism (not shown) to move the therapeutic bed 7-2 to the two-axis directions (X-axis direction and Y-axis direction) in a horizontal plane. The bed driving system 7-1 adjusts the position of the therapeutic bed 7-2 so that a diseased portion 5 of the patient 4 as a radiation field 5′ is located in an isocenter 5a in accordance with a diagnosis image imaged by the real time imager 30 (X-ray CT inspecting apparatus) under a control of a system control unit 8 which will be described later. The materials and shapes of the therapeutic bed 7-2 and the patient fixing unit 7-3 are selected to be suitable for the imaging process in a diagnosis image apparatus such as the real time imager 30, an X-ray solid imaging element device (X-ray CCD) and PET (Position Emission Tomography).

The X-ray head 10 irradiates a therapeutic X-ray 3a to the radiation field 5′ (diseased portion 5). This X-ray head 10 includes a small electronic linear accelerator for generating the therapeutic X-ray 3a having an energy of 4 MeV to 10 MeV. The X-ray head 10 is movably supported through a circumferentially moving mechanism 68 to the arc guide rail 9. Also, this X-ray head 10 includes the first swinging mechanism 131 and the second swinging mechanism 132.

The first swinging mechanism 131 swings (turns) the X-ray head 10 around a first swinging shaft S1 on the arc guide rail 9, as shown by an arrow R1 in FIG. 2. The first swinging shaft S1 is provided on the axis substantially passing through the inertia center of the X-ray head 10 or in the vicinity thereof, so that the inertia when the X-ray head 10 is swung is made small. The second swinging mechanism 132 swings (turns) the X-ray head 10 around a second swinging shaft S2 on the arc guide rail 9, as shown by an arrow R2 of FIG. 2. The second swinging shaft S2 is provided on the axis substantially passing through the inertia center of the X-ray head 10 or in the vicinity thereof, so that the inertia when the X-ray head 10 is swung is made small.

The arc guide rail 9 includes a guide rail inclining mechanism 28 and the circumferentially moving mechanism 68. The arc guide rail 9 is configured of a semi-circular ring having the shape of an upper half circle arc the therapeutic bed 7-2 and provided to stride the therapeutic bed 7-2. A guide rail inclining shaft 26 is a shaft in a Y-axis direction passing through both ends of the semi-circle and the center, and the center of the circle is coincident with the isocenter 5a. The arc guide rail 9 is inclinably supported by the guide rail inclining mechanism 28. The guide rail inclining mechanism 28 inclines the arc guide rail 9 in a range of 0° (the upright position in a Z-axis direction) and 90° (the position fallen in an X-axis direction) around the guide rail inclining shaft 26, as shown by an arrow G1 of FIG. 1. That is, the arc guide rail 9 can be moved to draw a quarter sphere (¼ spherical shell) with the isocenter 5a as a center. The arc guide rail 9 is made of a material having a strong rigidity such as a stainless steel.

Also, the circumferentially moving mechanism 68 circumferentially moves the X-ray head 10 on the semi-circle arc of the arc guide rail 9 along the arc guide rail 9, as shown by an arrow H1 of FIG. 2. A rack and opinion method or a belt method can be used for the circumferentially moving mechanism 68.

Through the foregoing three-shaft driving (G1, H1), the X-ray head 10 can carry out an isocentric motion, in which the X-ray head 10 is oriented towards the isocenter 5a, on the ¼ spherical shell with the isocenter 5a as the center. Moreover, through the foregoing two-shaft driving (R1, R2), the X-ray head 10 can carry out a pseudo non-isocentric motion, in which the X-ray head 10 is oriented towards a desirable point within a three-dimensional region 5b (refer to FIG. 2) in the vicinity of the isocenter 5a, on the ¼ spherical shell. This pseudo non-isocentric operation is the swinging motion around the inertia center of the X-ray head 10. Thus, as compared with the isocentric operation, the quick motion can be carried out at each stage. As the result of the tracking motion, which is pseudo non-isocentric, high responsive and quick, a radiation field of the X-ray head 10 can be tracked even to the quick motion such as the beat responsively and precisely.

The microwave generating unit 70 includes a klystron, a circulator 21 and a dummy load 22, which are related to a waveguide, and sends an electron accelerating microwave to the X-ray head 10 through the tracking type waveguide system 11. Here, the microwave of a C-band (for example, 5.6 GHz) is sent.

The tracking type waveguide system 11 is a waveguide for sending the microwave generated in the microwave generating unit 70 to the X-ray head 10. A joint section 14a, a link arm 12, a joint section 14b, a link arm 13, a joint section 14c, a link arm 15, a joint section 16 and the X-ray head 10 are linked to one after another to form a linking mechanism. Only the joint section 14a can be rotated around the shaft in the Y-axis direction. The joint section 14b, the joint section 14c and the joint section 16 can be rotated around the shaft in the X-axis direction. It should be noted that the X-ray head 10 at the link tip is slid along the arc guide rail 9 by the circumferentially moving mechanism 68 and swung around the joint section 16 by the first swinging mechanism 131. The joint sections 14a, 14b, 14c and 16 include a rotary RF coupler (not shown) for transferring the microwave through the shaft rotation. The link arms 12, 13 and 15 constitute a waveguides and are electro-magnetically connected through the joint sections 14a to 14c and 16. The microwave generated by the microwave generating unit 70 is sent through the joint section 14a—the link arm 12—the joint section 14b—the link arm 13—the joint section 14c—the link arm 15—the joint section 16 to the X-ray head 10.

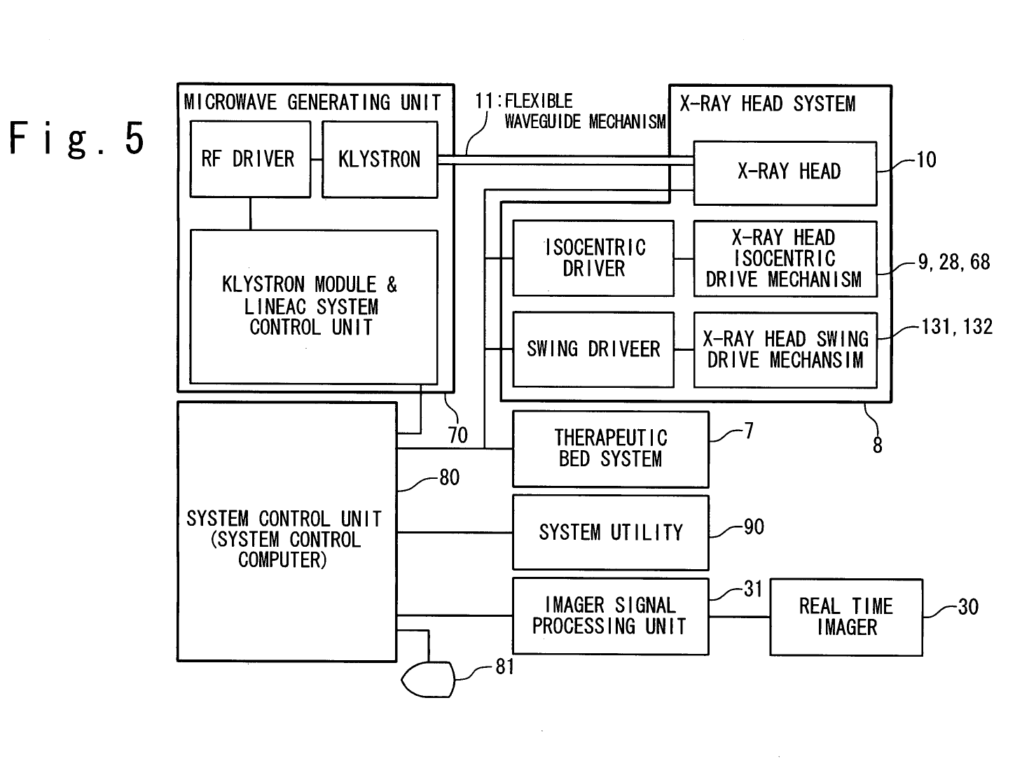

The real time imager 30 is an X-ray CT inspecting unit. The X-ray CT inspecting unit continuously irradiates a diagnostic X-ray 3b, which is a weak fan X-ray beam, to the diseased portion 5 of the patient 4 serving as the specimen from many directions over the entire circumference of 360 degrees and detects its transmission image. The detected diagnosis image is image-processed and consequently displayed on a display 81 as a three-dimensional tomography diagnosis image. The real time imager 30 is controlled by a system control unit 80 (FIG. 5). The real time imager 30 is supported in the attitude inclined at a predetermined angle (for example, an inclination of 20 to 30 degrees with respect to a vertical axis) by an imager inclining mechanism 20 shown in FIG. 1. When the imager inclining mechanism 20 is driven, the real time imager 30 is inclined around the shaft (represented by an arrow K1 of FIG. 1). Consequently, the radiation angle of the diagnostic X-ray 3b is changed. It should be noted that the real time imager 30 and the arc guide rail 9 are mechanically closely coupled and have a common coordinate system. This real time imager 30 is controlled to avoid the interference with the arc guide rail 9 and the X-ray head 10. When a usual X-ray camera is used as the imager, a small gold plate is embedded into the vicinity of the radiation field and used as a marker, according to necessary. The radiation field is indexed with this as a standard.

The real time imager 30 includes a donut-shaped vacuum chamber having a central opening as the diagnosis space. The patient 4 as the specimen together with the therapeutic bed 7-2 is inserted into and removed from this diagnosis space. The inside of the vacuum chamber is vacuum-exhausted through an exhaust port (not shown) by a vacuum pump.

In the vacuum chamber, a large number of diagnostic X-ray generating units are arrayed on a concentric circle near an outer circumference and a large number of sensor arrays are arrayed on a concentric circle near an inner circumference correspondingly thereto. These diagnostic X-ray generating units and the sensor arrays are shifted in the X-axis direction and arrayed. The diagnostic X-ray 3b is irradiated in the shape of a fan to the direction inclined against the radius of the vacuum chamber. Thus, the diagnostic X-ray 3b having the shape of the fan is not shielded by the sensor arrays on the X-ray radiation side (upper side) and can be transmitted through the patient 4 in the diagnosis space and detected by the sensor arrays on the opposite side (lower side).

Moreover, a beam limiter, an electronic gun driving circuit, an image signal digitizer and the like are provided in the respective proper positions of the vacuum chamber. The diagnostic X-ray 3b irradiated from the diagnostic X-ray generating unit is throttled by a collimator (not shown) and further defined to a width of a radiation position by the beam limiter. After being transmitted through the patient 4, the diagnosis X-ray is detected by the sensor arrays. The sensor arrays receive the diagnostic X-ray 3b having transmitted through the patient 4. They are densely fixed and provided on the circumference surrounding the diagnosis space where the patient 4 is placed, and have a large number of super high sensitive CdTe sensors and have a resolution of 0.5 mm. An imaging width of one shot at a time of the inspection is 80 mm. Also, a radiation time of the X-ray is 0.01 seconds per shot.

The X-ray transmission image detected by the sensor array is converted into a current signal proportional to the transmission X-ray amount, and sent through a pre-amplifier and a main amplifier to the image signal digitizer and the data recording unit, and then recorded as a diagnosis image data in the data recording unit. The imaging operation, the data recording operation and other operations of the diagnostic X-ray 3b are controlled by the system control unit 80. The recorded diagnosis image data is outputted from the data recording unit to an imager signal processing unit 31 (refer to FIG. 5) and data-processed by the imager signal processing unit 31. The processed data is reproduced and displayed as an X-ray CT diagnosis image of the diseased portion 5 on the display 81 of the system control unit 80.

On the other hand, a power source, an anode and a cathode of the diagnostic X-ray generating unit, and grid electrodes of a gate array are connected to an output side of an X-ray generation control unit of the real time imager 30, respectively. When the system control unit 80 outputs an X-ray generation command to the X-ray generation control unit, the X-ray generation control unit controls the power source to supply power to an electronic gun driving circuit from in response to the command, and also selects the grid electrode suitable for the imaging portion from the gate array. Thus, an electron beam is irradiated from any of the cathodes in the diagnostic X-ray generating unit. When a minus bias voltage applied to the selected grid electrode is released and becomes at a zero potential, the electron beam passes through the holes of the grid electrode and inputted to the anode. When the electron beam is inputted to the anode, a second X-ray is generated from the anode. In this way, the diagnostic X-ray 3b having the shape of the fan is irradiated through the collimator provided in a window towards the patient 4. It should be noted that the real time imager 30 does not need to be the X-ray CT inspecting unit and may be a set of the X-ray source and the sensor array opposite thereto.

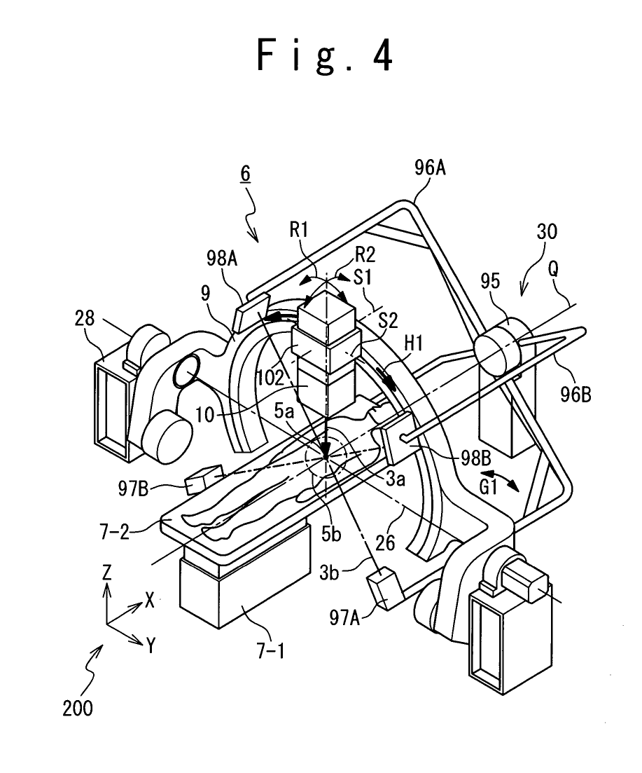

FIG. 4 is a perspective view of a radio-therapy apparatus employing a different real time imager 30. This real time imager 30 includes a rotation driving mechanism 95, holding frames 96A and 96B, and two sets of X-ray sources 97A and 97B and sensor arrays 98A and 98B for the usual X-ray cameras. The X-ray source 97A is provided at one end of the holding frame 96A, and the sensor array 98A is provided at the other end. The X-ray source 97B is provided at one end of the holding frame 96B, and the sensor array 98B is provided at the other end. The centers of the holding frames 96A and 96B are attached to the rotation driving mechanism 95.

The sensor array 98A is provided in the vicinity on one side in the Y-axis direction of the X-ray head 10. A perpendicular line from the center of the flat surface on the sensor side is oriented towards the isocenter 5a, and the X-ray source 97A is provided on its extension line. Similarly, the sensor array 98B is provided in the vicinity on the other side in the Y-axis direction of the X-ray head 10. The perpendicular line from the center of the flat surface on the sensor side is oriented towards the isocenter 5a, and the X-ray source 97B is provided on its extension line. The rotation driving mechanism 95 rotates the holding frames 96A and 96B around a real time imager rotation shaft Q which passes through the isocenter 5a and is parallel to the X-axis as the center, so that the two sets of the X-ray sources 97A and 98B are set to the desirable positions.

The two sets of the X-ray source and the sensor array 97A and 98A, and 97B and 98B are controlled to hold a predetermined angle therebetween. The predetermined angle is between 60 and 20 degrees in the route of the sensor array 98A or sensor array 98B—the isocenter 5a—the X-ray head 10. Preferably, it is between 45 and 30 degrees. This is set based on the condition that without any mutual influence between the X-ray head 10, the X-ray source 97A and the X-ray source 97B, they are accurately operated and the diagnosis image having a sufficient precision is obtained. However, the two sets of the X-ray source and sensor array 97A and 98A, and 97B and 98B may carry out the positional control independently of each other, unless the sight lines of the sets of the X-ray source and the sensor array are not coincident with each other.

In case of the radio-therapy apparatus shown in FIG. 4, the power source, anodes, cathodes and grid electrodes inside the X-ray sources 97A and 97B are connected to the output side of the X-ray generation control unit of the real time imager 30, respectively. When the system control unit 80 outputs the X-ray generation command to the X-ray generation control unit, the X-ray generation control unit controls the power source to supply power to an electronic gun driving circuit in response to the command, and operates the rotation driving mechanism 95 to move the two sets of the X-ray source and the sensor array to optimal positions on the basis of the positional relation to the X-ray head 10. In this case, the electron beam is irradiated from the cathodes inside the X-ray sources 97A and 97B, and the minus bias voltage applied to the grid electrode is released and becomes in the zero potential. Thus, the electron beam is passed through the holes of the grid electrode and inputted to the anode. When the electron beam is inputted to the anode, the second X-ray is generated from the anode. In this way, the diagnostic X-ray 3b having the shape of the fan is irradiated through the collimator provided in the window towards the patient 4.

The X-ray sources 97A and 97B are surely located on the sides opposite to each other with respect to a straight line connecting the X-ray head 10 and the isocenter 5a in FIG. 4. The sensor arrays 98A and 98B are also similar. Thus, the motions of the respective portions inside the patient 4 can be grasped quickly and accurately. Also, since the sensor arrays 98A and 98B are attached on the side of the X-ray head 10, the therapeutic X-ray 3a that is the very strong X-ray is never inputted to the sensor arrays 98A and 98B.

SAD (Source Axis Distance) shown in FIG. 1 corresponds to a distance from the isocenter 5a to an X-ray target (not shown) in the X-ray head 10. In this embodiment, the SAD serving as a reference is set to 80 to 100 cm.

A control system of the radio-therapy apparatus as mentioned above will be described below. FIG. 5 is a block diagram showing the configuration of this control system. This control system is provided with the therapeutic bed system 7, the X-ray head system 8, the real time imager 30, the imager signal processing unit 31, the microwave generating unit 70, the system control unit 80 and a system utility 90.

The system control unit 80 collectively controls the radio-therapy apparatus as a whole. This system control unit 80 contains a system control computer and includes [System Control Algorithm], [Image Tracking Algorithm], [Therapy Plan Algorithm], [Therapy Control Algorithm], [Graphical User Interface] and [Interlock Algorithm] as computer programs, and is provided with [Therapy Plan Database], [Trend Record Database] and [Therapy Database]. Also, the system control unit 80 includes a system monitor (display 81) and BIT. The system control unit 80 functions as a center machine, and other blocks are respectively connected to the system control unit 80 to input/output signals.

The [Therapy Plan Database] stores a therapy plan data as a data of therapy plan planned by a doctor. The therapy plan data is based on various inspections carried out prior to the radio-therapy. The therapy plan data relates a patient attribute data, a patient image data, an estimated absorption dose amount data, an therapeutic dose amount data, a diseased portion position data and the like.

The patient attribute data includes a name, a birth date and the like of the patient 4. The patient image data is configured of an X-ray tomography image of the patient 4. The estimated absorption dose amount data is a data of a dose amount of the radiation (X-ray) estimated to be absorbed in the diseased portion 5, and its irradiating method (the number of times, a dose amount estimated to be absorbed per one time and a radiation direction (route)), and indicates an estimated absorption dose amount setting. The therapeutic dose amount data is a data of a dose amount of the radiation (X-ray) absorbed in the diseased portion 5, and its irradiating method (the number of times, the absorbed dose amount per one time and the radiation direction (route)), and indicates the absorbed dose setting. The diseased portion position data is a data of the position of the diseased portion 5. The position of the diseased portion 5 may be a definition region 5-1 as described later.

The [Trend Record Database] stores a radiation result data as the result of the radio-therapy. The radiation result data is related to the radiation (X-ray) actually irradiated in the radio-therapy. The radiation result data relates the patient attribute data, the diseased portion image data, a therapeutic dose amount accumulation, an estimated absorption dose amount accumulation, a therapeutic dose amount for each radiation direction (portal number), an estimated absorption dose amount, a target coordinate (a coordinate of an radiation target in the diseased portion 5) and a mechanical coordinate (a coordinate of the radiation field 5′ on which the radiation is actually carried out). The diseased portion image data is the X-ray tomography image of the patient 4 obtained in real time from the real time imager 30 during the radio-therapy. A part of this diseased portion image data is reflected in the therapy plan database as the diseased portion image data. This radiation result data is provided to the doctor as a record of a therapy history and also filed in the Ministry of Health, Labor and Welfare according to a request.

The [Therapy Database] is used to relate a radiation absorption curve indicating a relation between kinds of substances, thicknesses of the substances, and an absorbed dose amount of the radiation (X-ray), and stores them.

The [System Control Algorithm] is used to collectively control the respective algorithms, GUI, the system monitor (display 81), BIT and the like in the whole of the system control unit 80.

The [Therapy Plan Algorithm] is used to calculate the therapeutic dose amount data (the therapeutic dose amount of the X-ray for each radiation direction (route), the therapeutic dose amount accumulation and the like) based on the therapy plan database (the X-ray tomography image of the patient 4, the estimated absorption dose amount data) and the therapy database (the radiation absorption curve for each substance). Then, the therapeutic dose amount data is displayed on the display 81 and checked by the doctor. The doctor changes the radiation direction and the estimated absorption dose amount of the X-ray and the like, as necessary, to set to the desirable therapeutic dose amount. After the check of the doctor, it is stored in the therapy plan database.

The [Therapy Control Algorithm] is used to control the X-ray head system 8 so that the X-ray head 10 is oriented towards a predetermined direction in accordance with the swinging amount of the X-ray head 10 from the therapy plan data of the therapy plan database and/or image tracking algorithm. Also, the [Therapy Control Algorithm] is used to store the radiation result data, which is obtained from the imager signal processing unit 31, the X-ray head system 8, the image tracking algorithm and the like during the therapy, in the trend record database.

The [Image Tracking Algorithm] is used to calculate the coordinate of the diseased portion 5 in accordance with the tracking image data obtained from the imager signal processing unit 31. Also, the [Image Tracking Algorithm] is used to determine the coordinate of the radiation field 5′ of the X-ray head 10 in accordance with various data obtained from the X-ray head system 8. Then, the [Image Tracking Algorithm] is used to calculate the swinging amount of the X-ray head 10 in accordance with the coordinate of the diseased portion 5 and the coordinate of the radiation field 5′.

The [Interlock Algorithm] is used to emergently stop the therapeutic X-ray 3a and the diagnostic X-ray 3b if a predetermined condition is satisfied. As the predetermined conditions, there are: a case that an emergency stop button is pushed; a case that the radiation field 5′ and the diseased portion 5 are separated by a preset distance or more; a case that at least one of the therapeutic dose amount and estimated absorption dose amount to the patient 4 exceeds an allowable value preset in correspondence to the dose amount; a case that the diagnostic X-ray 3b is stopped in the irradiation of the therapeutic X-ray 3a; and a case that the therapeutic X-ray 3a is stopped in the irradiation of the diagnostic X-ray 3b.

The X-ray transmission data detected by the real time imager 30 is re-configured to the diagnosis image by an image re-configuration algorithm in the imager signal processing unit 31 and sent to the system control unit 80. Thus, the diagnosis image is generated in real time during the radio-therapy. The doctor can carry out the radio-therapy while viewing the diagnosis image displayed on the display 81 of the system control unit 80.

The microwave generating unit 70 includes a klystron modulator & linear accelerator system control unit, a klystron and an RF driver. The klystron is connected to the X-ray head 10 through the tracking type waveguide system 11 and serves as a supply source for supplying microwave to an acceleration tube therein.

The X-ray head system 8 includes the X-ray head 10, the isocentric driving mechanism (having the arc guide rail 9, the guide rail inclining mechanism 28 and the head circumference moving mechanism 68) and the swing driving mechanism (having a first swinging mechanism 131, a second swinging mechanism 132 and a rotary RF coupler). The isocentric driving mechanism and the swing driving mechanism are connected to the system control unit 80 through the respective corresponding drivers (the isocentric driver and the swing driver) such that the head circumference moving mechanism 68 of the X-ray head 10 at the time of the isocentric radiation and the two-shaft swinging drive of the X-ray head 10 at the time of the pseudo isocentric radiation are respectively controlled.

The operation of the radio-therapy apparatus according to the embodiment of the present invention having the above-mentioned configuration will be described below with reference to the attached drawings.

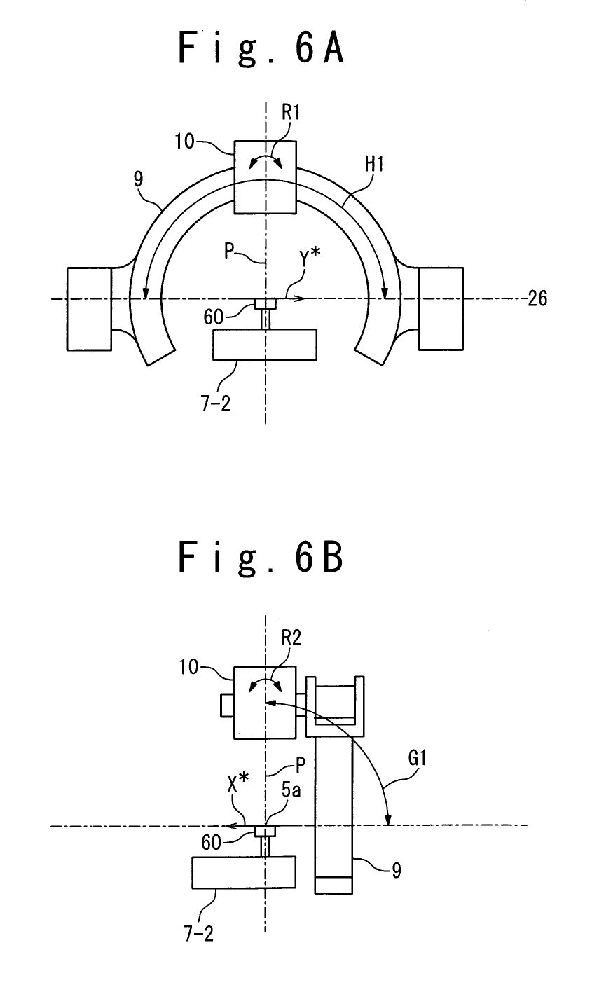

At first, the position calibration will be described. FIGS. 6A and 6B are views showing the position calibration of the radio-therapy apparatus 6, FIG. 6A shows a front view of the radio-therapy apparatus 6, and FIG. 6B shows a side view thereof. Other than the configurations shown in FIGS. 1 to 3, a CCD camera 60 is provided on the therapeutic bed 7-2.

The CCD camera 60 is provided such that the center of the light receiving surface thereof overlaps with the isocenter 5a, and the light receiving surface is in a horizontal plane. The CCD camera 60 is connected to a laser intensity analyzer (not shown). Instead of the acceleration tube and the like, a laser oscillator (not shown) such as a low output small He—Ne laser is provided in the X-ray head 10 so that it becomes coaxial with the irradiated X-ray.

The procedure of the position calibration will be described below.

(1) Step S1-1

In the situation shown in FIG. 6A, the laser oscillator of the X-ray head 10 outputs the laser to the CCD camera 60.

(2) Step S1-2

The CCD camera 60 receives the laser and then outputs a light reception result to the laser intensity analyzer (not shown).

(3) Step S1-3

The laser intensity analyzer detects an intensity distribution of the laser and then calculates the displacement (the X-axis direction, the Y-axis direction and the Z-axis direction) between the isocenter 5a (=the center of the light receiving surface of the CCD camera 60) and a peak position of the laser intensity.

(4) Step S1-4

The calculated displacement is stored as a correction value in a memory (not shown) of the system control unit 80.

By the method of the position calibration as mentioned above, a position displacement caused by distortion at a time of working, bending resulting from self-weight, stress at a time of attachment and the like in a large mechanical work piece can be corrected at an excellent precision in a very simple method in a short time. Thus, the position precision can be improved. In this embodiment, the position resolution can be improved to approximately 20 μm. This position calibration is carried out when the radio-therapy apparatus 6 is provided and periodically inspected. It should be noted that the position calibration may be carried out for each predetermined number of times of the uses or for each radio-therapy.

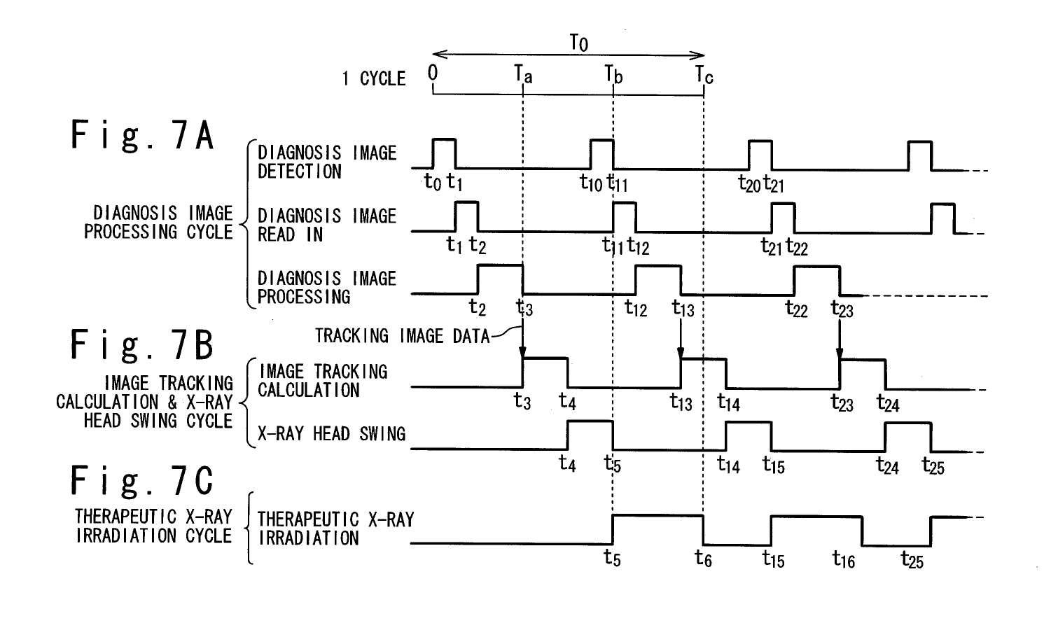

The operation of the radio-therapy apparatus according to the embodiment of the present invention will be described below with reference to timing charts shown in FIGS. 7A to 7C.

FIG. 7A is a timing chart showing timings when the diagnosis image is processed, FIG. 7B is a timing chart showing timings of the image tracking calculation based on the diagnosis image after the processing and the swinging operation of the X-ray head 10, and FIG. 7C is a timing chart showing the timing of the radiation of the therapeutic X-ray.

At first, when a main switch of the radio-therapy apparatus 6 is turned on, the power sources of the therapeutic bed system 7, the X-ray head system 8, the real time imager 30, the microwave generating unit 70, the system control unit 80 and the system utility 90 become in waiting states. Then, the therapeutic bed system 7 is actuated to move the patient 4 together with the therapeutic bed 7-2 to a therapy region. Then, the real time imager 30 is actuated to move the therapeutic bed 7-2 and to carry out the positioning so that the diseased portion 5 is coincident with the isocenter 5a of the therapy apparatus. In accordance with the following procedure, the real time image diagnosis is carried out by the real time imager 30, and the radio-therapy is carried out by the X-ray head 10, after the completion of this isocentric positioning.

(1) Step S2-1: Time t0 to t1

The real time imager 30 or the usual X-ray camera irradiates the diagnostic X-ray 3b from the diagnostic X-ray generating unit to the radiation field 5′. Then, the sensor array detects the X-ray transmission data as the diagnosis image data. In order to minimize the exposure, the radiation period of the diagnostic X-ray 3b is limited to t0 to t1.

(2) Step S2-2: Time t1 to t2

The detected diagnosis image data is converted into a current signal proportional to the transmission X-ray amount and then sent through the pre-amplifier and the main amplifier to the image signal digitizer and the data recording unit.

(3) Step S2-3: Time t2 to t3

The recorded diagnosis image data is sent from the data recording unit to the imager signal processing unit 31. Then, the imager signal processing unit 31 uses the image re-configuration algorithm, to carry out a calculation process on the diagnosis image data and to convert it into a tracking image data. The tracking image data is a data indicating the diagnosis image at the respective coordinate points (Xi, Yi, Zi), (i=1 to n: n is the number of the data) in the coordinate system of the radio-therapy apparatus 6. This tracking image data is sent to the system control unit 80.

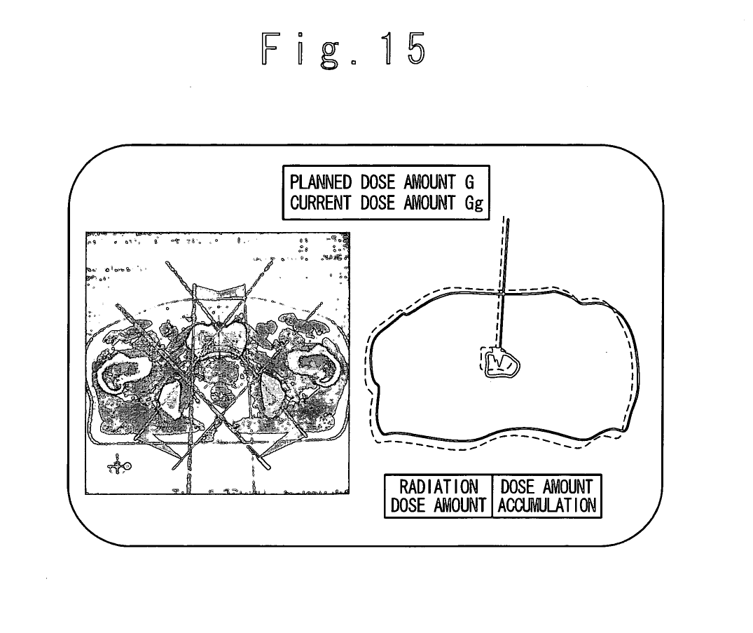

The system control unit 80 reproduces and displays the tracking image data as the (X-ray CT) diagnosis image of the diseased portion 5 on the display 81 and also stores as the diseased portion image data in the trend record database. In addition to the (X-ray CT) diagnosis image, a radiation dose amount and a dose amount accumulation are displayed on the display 81, as shown in FIG. 15. Consequently, the doctor can easily check the radio-therapy situation, determines whether or not the current radio-therapy is proper, and plans a next radio-therapy. It should be noted that the display on the display 81 may be configured to display the estimated absorption dose amount and the estimated absorption dose amount accumulation, in addition to the radiation dose amount and the dose amount accumulation. Also, an illustration on the right side of FIG. 15 shows that the therapeutic X-ray is irradiated to an actual radiation region (represented by the solid line) to a planed radiation region (represented by the dashed line).

The real time imager 30 and the imager signal processing unit 31 repeat the processes in the time t0 to t3 after the time t3 again. In FIG. 7A, the process at the time t3 is same as that of the time t10, and the processes in the time t0 to t3 are same as those of the time t10 to t13, the time t20 to t23, and the subsequent.

In order that the direct line, leakage line and dispersion line of the therapeutic X-ray 3a have no influence on the sensor array (detector) of the real time imager 30, the X-ray head 10 is interlocked such that the therapeutic X-ray 3a is not irradiated at least in the time period of the time t0 to t1 during which the diagnostic X-ray 3b is irradiated.

The total time t0 to t3 necessary for those diagnosis image processes (steps S2-1 to S2-3) is 0.01 seconds. That is, each cycle time of the diagnosis image process is 0.01 seconds. This is a sample rate sufficient to track the quick motion of the beat or the like.

(4) Step S2-4: Time t3 to t4

The system control unit 80 uses the image tracking algorithm to carry out the image tracking calculation as described below. That is, the system control unit 80 extracts the coordinate of the diseased portion 5 (the coordinate point (X, Y, Z) in the coordinate system of the radio-therapy apparatus 6) based on the tracking image data. On the other hand, the system control unit 80 calculates the coordinate of the radiation field 5′ of the current X-ray head 10 (the coordinate point (x, y, z) in the coordinate system of the radio-therapy apparatus 6) based on the position (coordinates), swing angles and the like of the guide rail inclining mechanism 28, head circumference moving mechanism 68, first swinging mechanism 131 and second swinging mechanism 132. Then, (i) if a distance L between two points=|(X, Y, Z)−(x, y, z)| is equal to or less than a preset value L02, the swinging control is determined not to be carried out, and (ii) if the distance L is equal to or greater than a preset value L01, the swinging angle is determined to be θ0, and (iii) if L02<the distance L<L01, the swinging angles (θ1, θ2) of the X-ray head 10 are calculated in accordance with the coordinate of the diseased portion 5 and the coordinate of the radiation field 5′.

However, the swinging angles (θ1, θ2) of the X-ray head 10 are a small displacement angle θ1 (of the rotation direction, a value of the rotation angle) around the S1 swinging drive shaft and a small displacement angle θ2 (of the rotation direction, the value of the rotation angle) around the S2 swinging drive shaft. The distance L01 is the maximum distance that the X-ray head 10 can carry out the swinging during the time t4 to t5. Also, the distance L2 is a value of an error that is estimated in the calculation of the coordinate point (X, Y, Z) of the diseased portion 5 and the coordinate point (x, y, z) of the radiation field 5′.

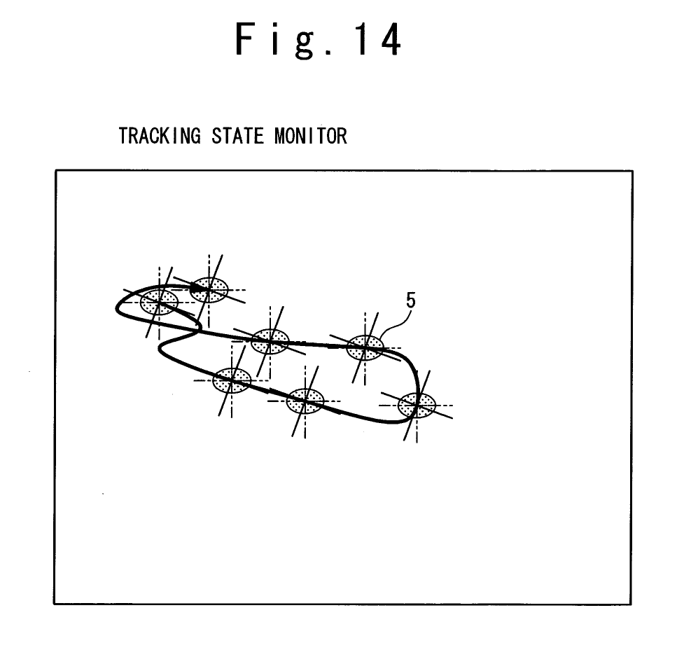

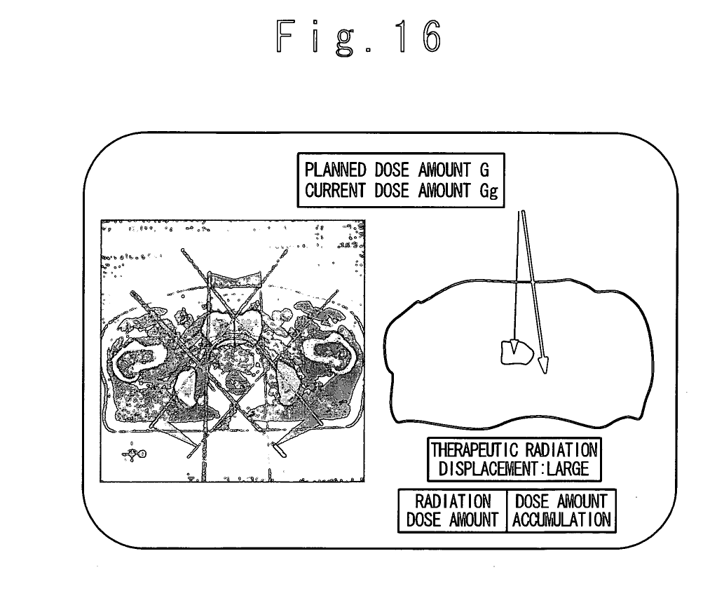

The situation of the movement (motion) of this diseased portion 5 (the coordinate point (X, Y, Z)) is displayed on the display 81 of the system control unit 80, as shown in FIG. 14. However, not only the diseased portion 5 but also its peripheral region (for example, a boarder line 5-2 (which will be described later) including the diseased portion 5) may be similarly displayed. At this time, if the direction of the actual dose amount (indicated by the solid line) is greatly displaced from the planed dose amount (indicated by the dashed line) as represented in the image on the right side in FIG. 16, a message of [therapeutic Radiation Displacement: Large] is displayed, and the interlock is also carried out. Consequently, the doctor can determine whether or not the radio-therapy is proper in accordance with the total data including even the situation of the therapy portion.

(5) Step S2-5: Time t4 to t5

In accordance with the calculated swinging angles (θ1, θ2) of the X-ray head 10, the system control unit 80 uses the therapy control algorithm to send a swinging drive signal indicative of the swinging angles (θ1, θ2) of the X-ray head 10 to the X-ray head system 8. In accordance with the swinging drive signal, an X-ray head swinging driver of the X-ray head system 8 drives the first swinging mechanism 131 and the second swinging mechanism 132, such that the X-ray head 10 is oriented towards the desirable direction. The system control unit 80 again repeats the processes of the time t3 to t5 from the time t13 after the time t5. In FIG. 7B, the processes during the time t3 to t5 are same as those during the time t13 to t15, the times t23 to t25, and the subsequent.

The total time t3 to t5 necessary for those image tracking calculation and X-ray head swinging (steps S2-4, S2-5) is 0.01 seconds. That is, one cycle time of the image tracking calculation and X-ray head swinging is 0.01 seconds. This is a rate sufficient to track the quick motion of the beat or the like.

In the time t4 to t5 during which an S1 swinging drive servo motor of the first swinging mechanism 131 and an S2 swinging drive servo motor of the second swinging mechanism 132 (both of them are not shown) are driven, there may be a possibility of an error operation of the swinging angle. Therefore, the X-ray head 10 is interlocked to insure the safety in such a way that the therapeutic X-ray 3a is not irradiated.

(6) Step S2-6: Time t5 to t6

The system control unit 80 uses the system control algorithm to send a therapeutic X-ray radiation signal to the X-ray head 10 as a signal for instructing the radiation of the therapeutic X-ray 3a at the time t5. Consequently, the interlock of the X-ray head 10 is released and irradiation of the therapeutic X-ray 3a to the diseased portion 5 is started. The radiation time t5 to t6 of the therapeutic X-ray 3a is about 5 ms. The duty of the radiation is about 50%. The system control unit 80 again repeats the process during the time t5 to t6 from the time t15 after the time t6. In FIG. 7C, the processes during the time t5 to t6 is same the processes during the time t15 to t16, the time t25 to t26, and the subsequent.

A total time t5 to t6 necessary for this therapeutic X-ray radiation (step S2-6) is 0.01 seconds. That is, one cycle time of the therapeutic X-ray radiation is 0.01 seconds. This is the sufficient rate to track the quick motion of the beat or the like.

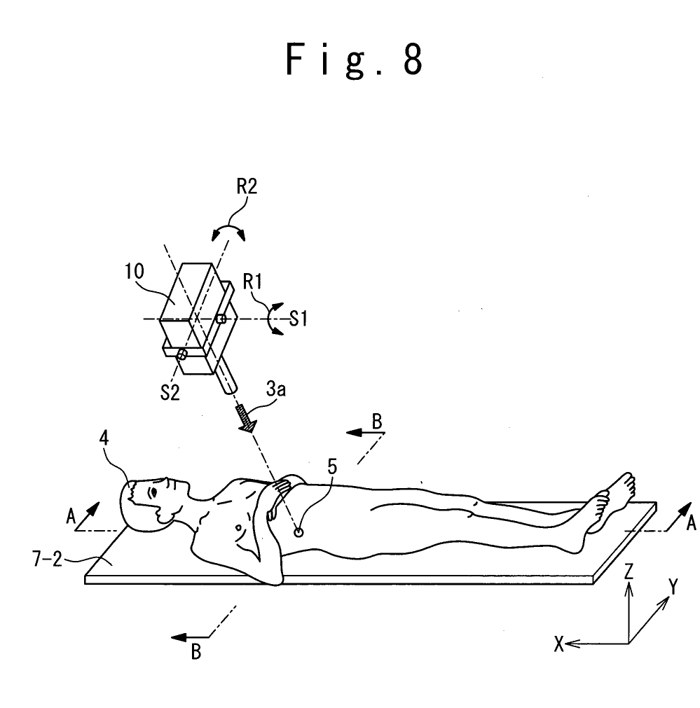

Here, the manner when the therapeutic X-ray 3a is irradiated while the X-ray head 10 is swung will be further described with reference to the drawings. FIG. 8 is a perspective view showing a manner in the radio-therapy using the X-ray head 10. The X-ray head 10 irradiates the X-ray to the diseased portion 5.

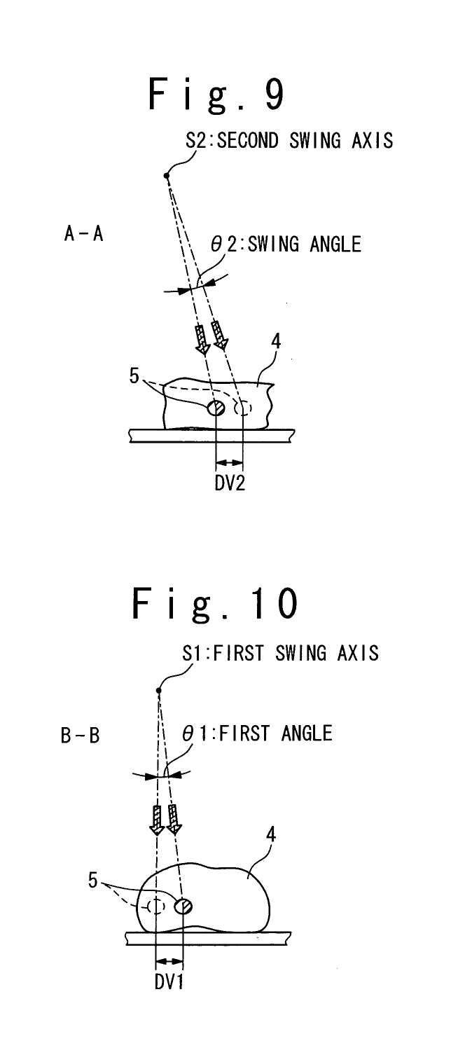

FIGS. 9 and 10 are views showing the manner when the therapeutic X-ray 3a is irradiated while the X-ray head 10 is swung. FIG. 9 is the A-A section in FIG. 8, and FIG. 10 is the B-B section in FIG. 8.

In order to carry out the radiation while tracking the movement of the radiation field, the system control unit 80 calculates shift amounts DV1 and DV2 from the radiation field 5′ of the diseased portion 5 in the X-axis and Y-axis directions in accordance with the calculated positions (the coordinate (X, Y, Z)) of the diseased portion 5 and the current coordinate (x, y, z) of the radiation field 5′ of the X-ray head 10. Then, in accordance with the shift amounts DV1 and DV2, a predetermined calculation equation is used to determine the displacement angles θ1 and θ2 resulting from the movements around the first swing drive shaft S1 and second swing drive shaft S2, respectively.

In the foregoing time t5 to t6, the X-ray head 10 is fast swung by the displacement angle θ1 around the first swinging drive shaft S1 and by the displacement angle θ2 around the second swinging drive shaft S2. Then, simultaneously with the stop of the swinging operation, the X-ray head 10 irradiates the therapeutic X-ray 3a.

Through the above-mentioned steps S2-1 to S2-6, the collimation of the X-ray head 10 can track the diseased portion 5 such as the tumor or the like, which is moved by the influence of the motions and situations of organs such as the respiration and beat below the neck, the peristalsis, the urine amount inside the bladder, and the like, quickly and high responsively, and irradiates the radiation (X-ray) at a high precision. That is, it is possible to carry out the swinging operation on the X-ray head 10 at the high speed within 0.03 seconds including the processing time of the diagnosis image and possible to quickly track the motion of the radiation field (diseased portion).

In the foregoing process, at the Step S2-4: the time t3 to t4, the angle at which the X-ray head 10 is swung is limited to a predetermined value at the step S2-5. This reason is as follows. That is, when the swinging angle becomes larger, the time necessary for the swinging becomes longer. Meanwhile, the diseased portion 5 is further moved. As a result, the coordinate point (x, y, z) of the radiation field 5′ of the X-ray head 10 is largely displaced from the position of the coordinate point (X, Y, Z) of the diseased portion 5.

The quick motion of the diseased portion 5 tracked by the X-ray head 10 results from the respiration and the beat. In this case, the diseased portion 5 is moved within the substantially same region (however, the routes are not always same). Thus, even if there is a case that the coordinate point (x, y, z) of the radiation field 5′ of the X-ray head 10 is not perfectly coincident with the coordinate point (X, Y, Z) of the diseased portion 5 at a time, they can be made coincident after that.

If an abnormality occurs in the obtainment of the diagnosis image data or the image tracking calculation, the interlock is carried out on the radiation of the therapeutic X-ray 3a at that time, to stop the radiation, thereby insuring the safety. This apparatus is designed so as to irradiate the therapeutic X-ray 3a after the normal executions of the swinging and positioning of the X-ray head 10 are checked. Then, if a difference between the coordinate point (x, y, z) of the radiation field 5′ and the coordinate point (X, Y, Z) of the diseased portion 5 is equal to or greater than a preset allowable value, the radiation of the therapeutic X-ray 3a in the step S2-6 (the time t5 to t6) is not carried out in that cycle.

Also, the system control unit 80 can move the head circumference moving mechanism 68, the inclining mechanism 28 and the therapeutic bed system 7, as necessary, to coincide the collimation of the X-ray head 10 with the diseased portion 5. In this case, the system control unit 80 calculates the swinging amount (for the first and second swinging mechanisms 131 and 132) and movement amount (for the head circumference moving mechanism 68, the inclining mechanism 28 and the therapeutic bed system 7) of the X-ray head 10 in accordance with the coordinate of the diseased portion 5 and the coordinate of the radiation field 5′ during the time t3 to t4. Subsequently, during the time t4 to t5, the system control unit 80 outputs the swinging amount and movement amount of the X-ray head 10 to the X-ray head system 8. Then, the first and second swinging mechanisms 131 and 123, the head circumference moving mechanism 68, the inclining mechanism 28 and the therapeutic bed system 7 are driven to match the collimation of the X-ray head 10 with the diseased portion 5.

After the radiation stop of the therapeutic X-ray 3a, at the time t5, the radiation of the diagnostic X-ray 3b is started to proceed to the next diagnosis image process cycles t5 to t8. Subsequently, at the time t3 after the diagnosis image process, the interlock of the X-ray head 10 is released to resume the radiation of the therapeutic X-ray 3a.

In this way, the cycle of the total 0.03 seconds is repeated, which is composed of: the diagnosis image process cycle (0 to Ta in FIGS. 7A to 7C) of 0.01 seconds; the image tracking calculation cycle and the X-ray head swinging cycle (Ta to Tb in FIGS. 7A to 7C) of 0.01 seconds; and the therapeutic X-ray radiation cycle (Tb to Tc in FIGS. 7A to 7C) of 0.01 seconds. That is, the X-ray head can be accurately oriented towards the radiation target for each approximately 1/30 seconds. Even if the diseased portion (therapy field) has the quickest motion such as the beat, it is possible to track the radiation target accurately in the real time and to irradiate the radiation.

Also, the diagnosis image data of the diseased portion 5 that is the tracking image data under the therapy are sequentially stored as the diseased portion image data in the trend record database. Thus, the portion to which the therapeutic radiation is actually irradiated can be clearly known by referring to this trend record database in future. Therefore, the diseased portion image data is sufficient as the record of the therapy history, and it is easy to plan the therapy plan after that.

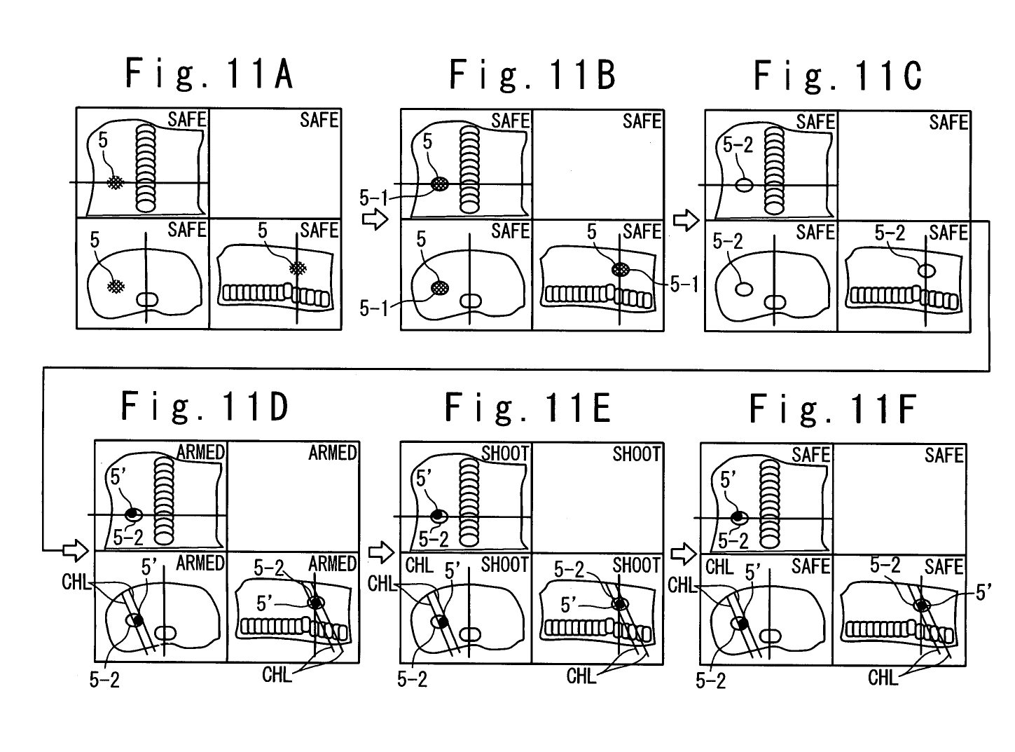

The procedure of a pseudo non-isocentric therapy will be described below. FIGS. 11A to 11F are flowcharts showing the procedure of the pseudo non-isocentric therapy with the displaying on the display 81. It should be noted that in examples shown in FIGS. 11A to 11F, the diagnosis images of the diseased portion from the three directions of X, Y and Z are displayed on the display 81.

(1) Step S3-1

In the radio-therapy, the doctor prepares a therapy plan. The therapy plan is based on various inspections carried out prior to the operation. This therapy plan is stored in the therapy plan database. Moreover, by using the radio-therapy apparatus during the operation, the doctor can directly carry out the image diagnosis on the diseased portion in real time and consequently execute the radio-therapy at a high precision and a high sureness.

(2) Step S3-2

As shown in FIG. 11A, only the real time imager 30 and the imager signal processing unit 31 are used to re-configure the diagnosis image of the diseased portion 5 and peripheral region thereof. Then, the re-configured image is reproduced and displayed on the display 81 of the system control unit 80. In this case, as shown in FIG. 15, not only the radiation dose amount and the accumulation of dose amounts but also the estimated absorption dose amount and the estimated absorption dose amount accumulation are displayed on the display 81. However, they are omitted in FIGS. 11A to 11F. The re-configuration of the diagnosis image is carried out in the foregoing steps S2-1 to S2-3. However, at this stage, the steps S2-4 to S2-6 are not carried out.

(3) Step S3-3

As shown in FIG. 11B, the doctor checks the respective sectional views of the diseased portion 5 on the display 81 and defines the contour of the radiation field 5′ to track the image. Here, prior to the radio-therapy start, the mapping of the radiation field 5′ has been already ended (the therapy plan database). Then, with reference to this, the contour of the radiation field 5′ is defined in a plurality of slices. The region defined in the contour is a definition region 5-1, and the definition region 5-1 contains the diseased portion 5. The definition region 5-1 is stored in the therapy plan database.

The therapy plan algorithm is used to calculate the therapeutic dose amount data (the therapeutic dose amount of the X-ray for each radiation direction (route), the therapeutic dose amount accumulation and the like) in accordance with the therapy plan database (including the definition region 5-1) and the therapy database. Then, the calculated therapeutic dose amount data is displayed on the display 81 and checked by the doctor. As necessary, the doctor changes the radiation direction, the estimated absorption dose amount of the X-ray and the like so that it becomes the desirable therapeutic dose amount data. After the check of the doctor, the therapeutic dose amount data is stored in the therapy plan database.

(4) Step S3-4

As shown in FIG. 11C, the image tracking algorithm of the system control unit 80 is used to extract the image contour. That is, the pattern matching between the actual diagnosis image of the diseased portion 5 and the defined contour of the definition region 5-1 is carried out, and the pattern matching result is displayed as the contour 5-2 (which will be described later). Then, the image tracking is started. As shown in FIG. 14, the movement situation of the diseased portion 5 is displayed on the display 81. The doctor visually checks the image tracking situation. The image tracking is carried out at the step S2-4. Thus, the steps S2-1 to S2-4 are repeated. However, at this stage, the steps S2-5 to S2-6 are not carried out.

(5) Step S3-5

As shown in FIG. 11D, after the image tracking becomes stable, the doctor operates a master arm switch to set the X-ray head system 8 in an ARMED state. The X-ray head system 8 displays a collimation in a cross hair line and a radiation volume in a red color on the display 81. Then, simultaneously with the image tracking, the tracking (swinging) of the X-ray head 10 is carried out. Since the tracking of the X-ray head 10 and image is continuous, the collimation and the radiation volume automatically track together with the movement of the radiation field 5′. The tracking (swinging) of the X-ray head 10 is carried out at the step S2-5. Thus, the steps S2-1 to S2-5 are repeated. However, since the radiation of the therapeutic X-ray 3a is not carried out at this stage, the step S2-6 is not carried out.

(6) Step S3-6

As shown in FIG. 11E, a triggering operation of the doctor starts radiation of the therapeutic X-ray 3a. At the stage of the therapy plan, a scheduled radiation time is determined, and countdown is started on the display 81. On the other hand, the radiation time of one radiation (Step S2-6: Time t5 to t6) is also determined. Thus, the count is reduced while the radiations in a short time (time t5 to t6) are repeated. Then, when it becomes finally zero, the therapeutic X-ray 3a is automatically stopped. The therapeutic dose amount of the therapeutic X-ray 3a is detected by an ionization chamber (not shown) inside the X-ray head 10 and outputted to the therapy control algorithm. The radiation of the therapeutic X-ray 3a is carried out at the step S2-6. Therefore, the steps S2-1 to S2-6 are repeated.

Also, by the therapy control algorithm, (a part or all of) the radiation result data obtained from the imager signal processing unit 31, X-ray head system 8, image tracking algorithm and the like during the radio-therapy are continuously displayed on the display 81. The doctor continues to trigger for continuous radiation, while checking (the part or all of) the radiation result data. The radiation result data is stored in the trend data database.

The system control unit 80 continues to alternately to sample (track) the diagnosis image and to irradiate the therapeutic X-ray 3a at a high speed and continues to carry out the tracking of the image and the radiation of the therapeutic X-ray in the real time. Even before the countdown becomes zero, if the doctor releases the trigger, the therapeutic X-ray 3a is stopped immediately at that timing. Thus, the safety is sufficiently insured.

(7) Step S3-7

As shown in FIG. 11F, the doctor sets the master arm switch at a SAFE position to keep the system safe and moves the X-ray head 10 to a next radiation position. At this stage, the steps S2-1 to S2-3 are carried out, but the steps S2-4 to S2-6 are not carried out.

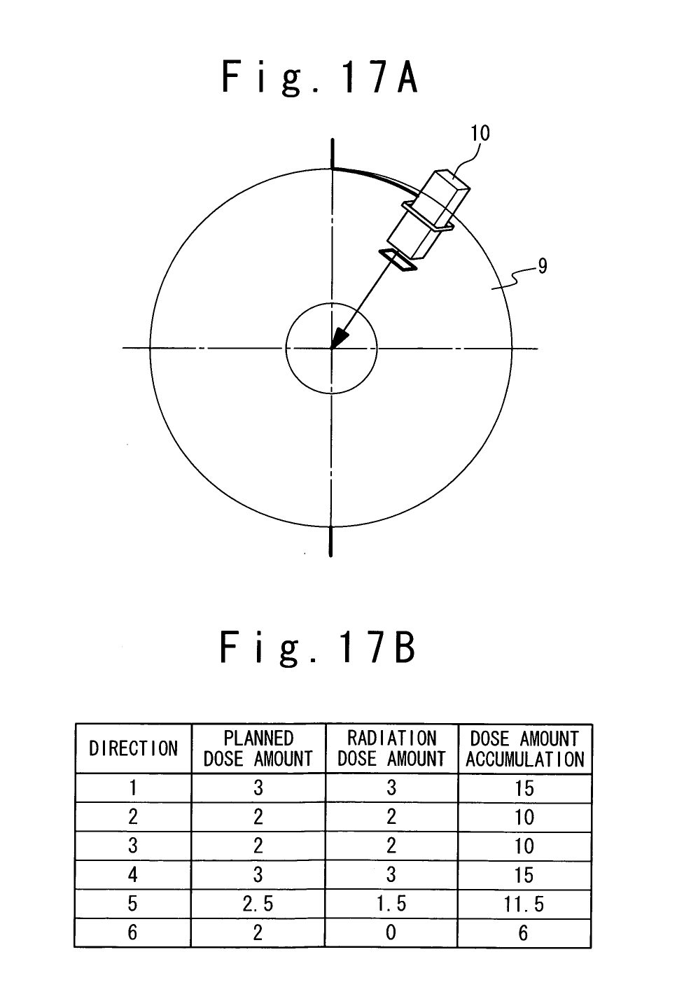

The foregoing steps S3-1 to S3-7 are carried out on a plurality of radiation directions (coordinates). The radiation result data for each radiation direction obtained from the two diagnosis images is stored in the trend record database. Specifically, as conceptually shown in FIG. 17A, the three-dimensional coordinate is determined such that the XY-axis direction is determined on the basis of the direction of the radiation from the X-ray head 10, and the Z-axis direction is determined on the basis of the inclination of the arc guide rail 9. FIG. 17B shows an example that the planed dose amount, therapeutic dose amount and accumulation of dose amounts with regard to six radiation directions are stored in the trend record database. By this configuration, the coordinate data of the diseased portion of the patient is obtained, which makes the radiation direction clearer and makes the check with the therapy plan easier.

After the end of the series of the radiations, the doctor checks the total dose amount as a summation of the accumulated exposure dose amounts per day. That is, the therapy control algorithm is used to read out the data from the trend record database and to display the accumulation of dose amount of that day and a distribution of the accumulations of dose amounts within one course on an image screen. The data with regard to the radio-therapy is stored in the therapy file (including the radiation result data) prepared for each patient 4, in the trend record database.

Here, the method of matching the pattern between the actual diagnosis image of the diseased portion 5 and the contour of the definition region 5-1 at the step S3-4 will be further described.

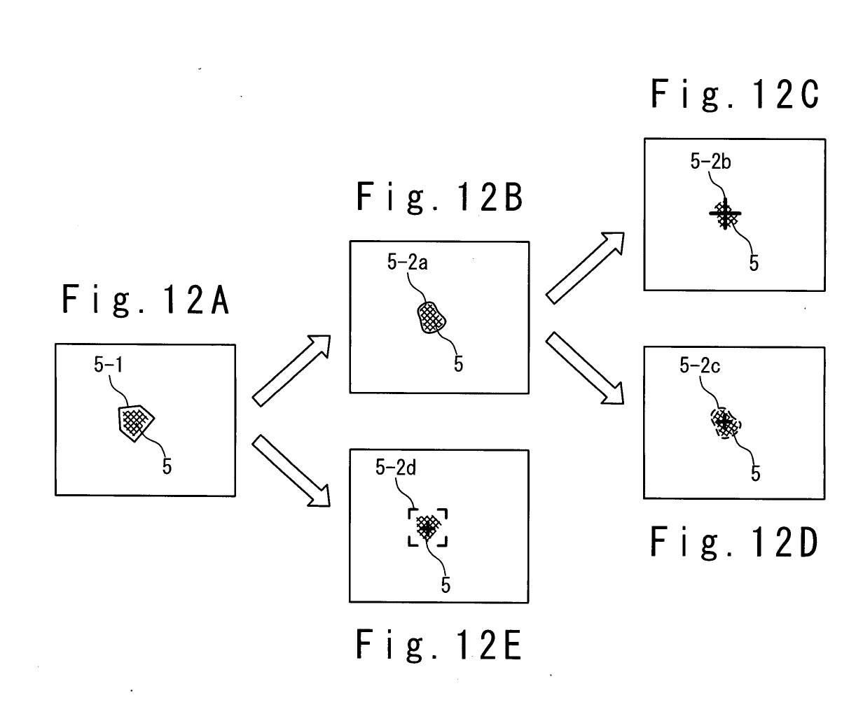

FIGS. 12A to 12E are diagrams showing the relation between the diseased portion 5, the definition region 5-1 and the contour 5-2 resulting from the pattern matching. FIG. 12A shows the relation between the diseased portion 5 and the definition region 5-1, and FIGS. 12B to 12E show the relation between the diseased portion 5 and the contour 5-2.

(1) Step S4-1

As shown in FIG. 12A, the doctor uses a touch pen with which it can be drawn on the display 81 or a pointer such as a mouth and indicates the definition region 5-1 on the display 81 by way of a drawing tool.

(2) Step S4-2

The therapy plan algorithm is used to extract the diagnosis image within the definition region 5-1 in accordance with the definition region 5-1 drawn on the display 81 and the diagnosis image on the display 81. Thus, the shape, coordinate and brightness distribution of the diagnosis image are grasped. Or, by extracting the shape of the brightness distribution occupying a predetermined rate (for example, 90%) of the definition region 5-1 as shown in FIG. 12B, the shape, coordinate and brightness distribution of the diagnosis image are grasped.

(3) Step S4-3

The therapy plan algorithm is used to determine the gravity-center, with regard to the shape of the definition region 5-1, or the shape of the brightness range indicating the predetermined rate. Then, the gravity-center is displayed as a “+” symbol on the display 81. For example, the gravity-center of the definition region 5-1 (FIG. 12A) is as shown in FIG. 12C. The gravity-center of the brightness range (FIG. 12B) indicating the predetermined rate is as shown in FIG. 12D. It should be noted that as shown in FIG. 12E, only the gravity-center may be merely shown. As mentioned above, the pattern matching is ended.

On the display 81, the binary display can be carried out such that the range of the definition region 5-1 or the brightness range indicating the predetermined rate is represented by a particular color, and the others are represented by the other colors. Thus, the definition region 5-1 and the like can be easily discriminated.

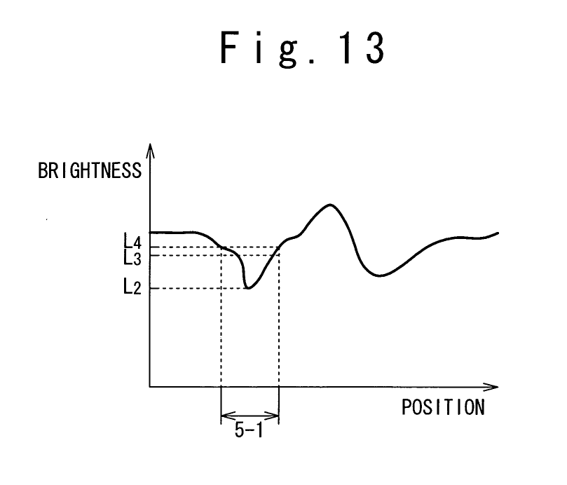

However, the brightness distribution is grasped as described below. FIG. 13 is a graph showing one example of the brightness distribution in the diagnosis image. The vertical axis indicates the brightness, and the horizontal axis indicates the position of the diagnosis image. The brightness in the definition region 5-1 of the diagnosis image is known to be in a range between L2 and L4 from the graph. Thus, the brightness range of the definition region 5-1 is between L2 and L4. Also, a brightness region occupying a predetermined rate (for example, 90%) of the definition region 5-1 is a continuous brightness range between L3 and L4, which is selected so as to occupy the region of the predetermined rate (for example, 90%) of the definition region 5-1. It should be noted that a different position indicating the same brightness is separated from the definition region 5-1. Thus, the different position is not recognized as the definition region 5-1.

As described above, according to the radio-therapy apparatus according to the embodiment of the present invention, it is possible to carry out the high speed swinging operation on the radiation head (X-ray head 10) within 0.02 seconds including the imaging process and possible to track the motion of the radiation field (diseased portion). Thus, the radiation can be irradiated (at the radiation time of 0.01 seconds) at the high precision. In this way, in response to the motion of the diseased portion, the non-isocentric radiation can be carried out at the high response and at the high precision. Therefore, the portion that the radiation target is moved by the influence of the motions and situations of the organs such as the respiration and beat below the neck, the peristalsis, the urine amount inside the bladder, and the like can be targeted for the radio-therapy. It should be noted that in the foregoing example, as the inspecting unit, a combination of the real time imager 30 and the radio-therapy apparatus was described. However, the present invention is not limited only to this. A combination of the usual X-ray camera and a different non-magnetic inspecting unit such as PET (Position Emission Tomography) and the like in a special application field can be combined with the radio-therapy apparatus.

When the usual X-ray camera is used, two or more cameras having different sight lines are required. Also, a soft tissue having a low contrast and the like cannot be imaged. Thus, the X-ray CT, the MRI or the like is preliminarily used to make it possible to position the radiation field based on a land mark having a high contrast such as a bony tissue and the like. Or, a small gold plate is embedded into the vicinity of the radiation field and used as the marker. Or, a devise is considered to make it possible to emphasize the image by using a contrast medium or differential image process such as DSA (Digital Subtraction Angiography). Also, in the X-ray CT and the PET, the real time image re-configuration calculation of a high speed is carried out for the real time imaging.

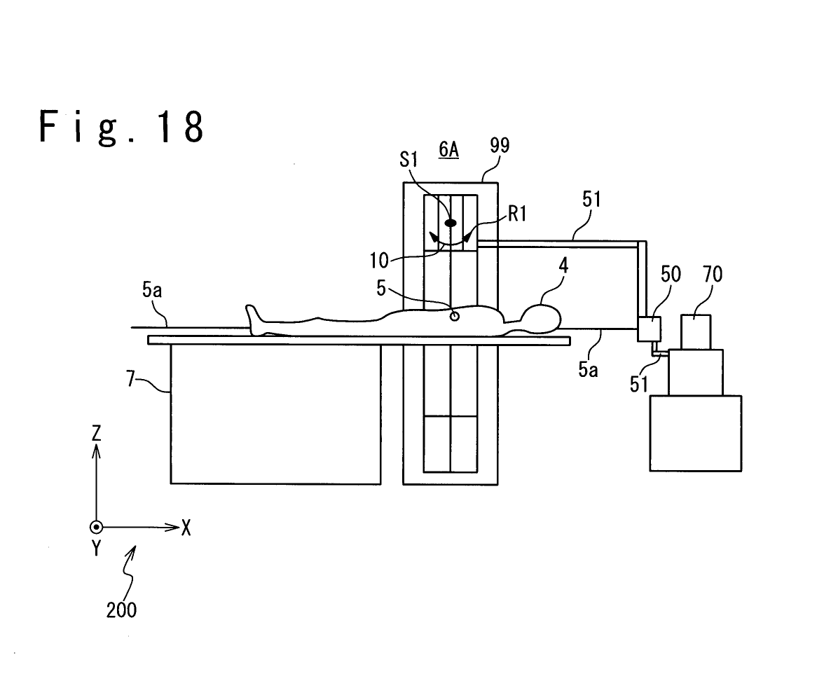

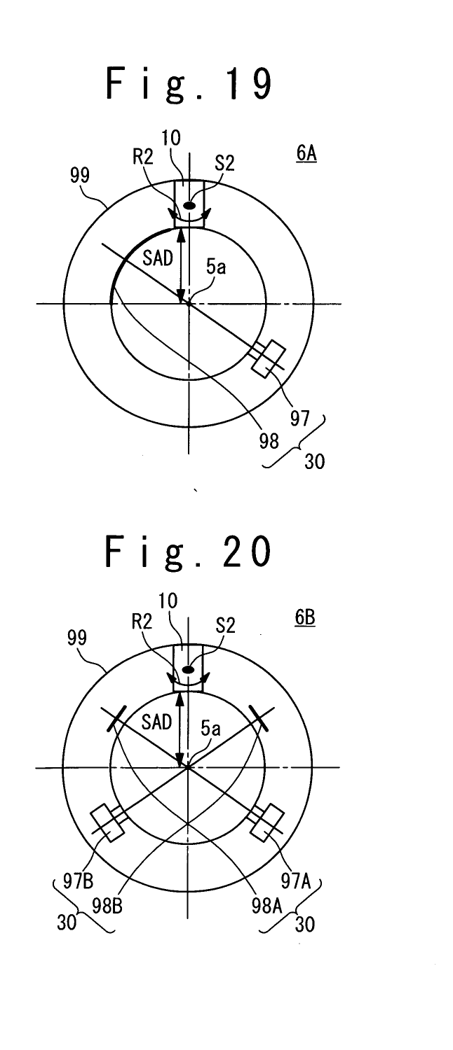

A first modification example of the radio-therapy apparatus according to the embodiment of the present invention will be described below with reference to FIGS. 18 and 19. It should be noted that the description of a part overlapping with the description of the radio-therapy apparatus according to the foregoing embodiment is omitted.

FIG. 18 is a side view showing the configuration of the first modification example of the radio-therapy apparatus according to the embodiment of the present invention, and FIG. 19 is a front view showing the configuration of its rotating drum (therapy gantry).

In this radio-therapy apparatus 6A, the therapeutic X-ray head 10, a therapeutic X-ray source (CT X-ray tube) 97 and a sensor array 98 are provided on a rotating drum (treating gantry) 99. That is, as the structure of the entire apparatus, the X-ray head 10 is provided on the upper portion of the drum portion of the rotational type X-ray CT inspection apparatus that is the real time imager 30 in the foregoing embodiment. The rotation center of the rotating drum (treating gantry) 99 is the isocenter 5a. The X-ray head 10 is configured of the electric linear accelerator of 4 MeV to 10 MeV. As illustrated, the X-ray head 10 can be swung around the two shafts (the first and second swinging shafts S1 and S2). That is, through those swinging operations, the non-isocentric radiation in the two shafts is made possible in addition to the isocentric radiation around the rotation shaft of the rotating drum. It should be noted that the swinging around the second swinging shaft S2 includes an X-ray head swing angle correction associated with the rotation of the rotating drum.

The therapeutic X-ray source (CT X-ray tube) 97 and the sensor array 98 are attached to the positions which do not bring about the interference with the therapeutic X-ray head 10, respectively. The therapeutic X-ray source (CT X-ray tube) 97 and the sensor array 98 face each other. The sensor array 98 for the detection is used for the X-ray, and this is a multi-row sensor of a multi-array type. In the X-ray CT and the PET, the real time image re-configuration calculation process of the high speed is carried out on the real time imaging.

A second modification example of the radio-therapy apparatus according to the embodiment of the present invention will be described below with reference to FIG. 20. It should be noted that the description of a part overlapping with the description of the radio-therapy apparatus according to the foregoing embodiment and first variation example is omitted.

FIG. 20 is a front view showing the configuration of the rotating drum (treating gantry) in the second modification example of the radio-therapy apparatus according to the embodiment of the present invention. In this radio-therapy apparatus 6B, the therapeutic X-ray head 10 and the two sets of the X-ray sources 97A and 97B and the sensor arrays 98A and 98B are provided on the rotating drum (treating gantry) 99. Those relative positions are fixed within a predetermined range. The predetermined range is between 60 and 20 degrees, with regard to the angle between the sensor array 98A or the sensor array 98B, the isocenter 5a and the X-ray head 10. Preferably, it is between 45 and 30 degrees. This is determined in accordance with the condition that without any mutual influence between the X-ray head 10, the X-ray sources 97A and the X-ray source 97B, each of them is accurately operated and the diagnosis image having the sufficient precision is obtained.

Unlike the radio-therapy apparatus of the first modification example that includes the therapeutic X-ray source (CT X-ray tube) and the sensor array, the rotating drum 99 includes the two sets of the X-ray sources 97A and 97B and the sensor arrays 98A and 98B provided so that the sight lines of the sets of the X-ray source and the sensor array are not in coincidence with each other. The X-ray sources 97A and 97B are located on the side opposite to each other, with respect to the straight line between the isocenter 5a and the X-ray head 10 in FIG. 20. The sensor array 98A and the isocenter 98B are similar.

Consequently, it is possible to obtain the X-ray transmission images such as the diseased portion 5, the land mark, the micro gold metal and the like within the body of the patient 4 in the two shafts, and also possible to grasp the motions of the respective portions within the body of the patient 4 quickly and accurately. It should be noted that as the image emphasizing method of the X-ray transmission image, the method will be considered for using contrast medium to carry out the image process such as DSA. Also, since the sensor arrays 98A and 98B are provided on the side of the X-ray head 10, the therapeutic X-ray 3a which is the very strong X-ray is never inputted to the sensor arrays 98A and 98B.

The X-ray head 10 is configured of the electric linear accelerator of 4 MeV to 10 MeV. As illustrated, the X-ray head 10 can be swung around the two shafts (the first and second swinging shafts S1, S2). That is, through those swinging operations, the non-isocentric radiation around the two shafts can be made possible, in addition to the isocentric radiation around the rotation shaft of the rotating drum. It should be noted that the swinging of the second swinging shaft S2 includes the X-ray head swing angle correction associated with the rotation of the rotating drum.

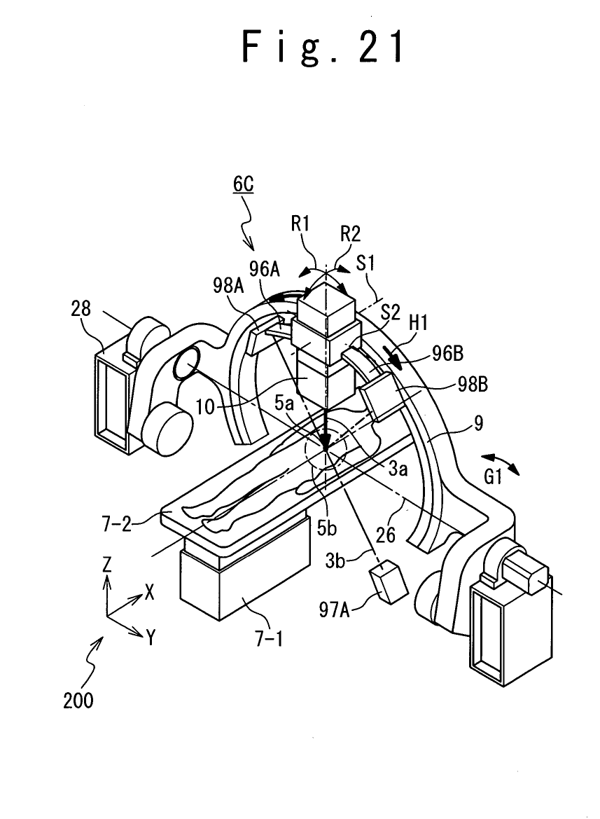

A third modification example of the radio-therapy apparatus according to the embodiment of the present invention will be described below with reference to FIG. 21. It should be noted that the description of a part overlapping with the description of the radio-therapy apparatus according to the foregoing embodiment and first and second modification examples is omitted.

FIG. 21 is a perspective view showing the configuration of the radio-therapy apparatus according to the third modification example of the embodiment of the radio-therapy apparatus in the present invention. This radio-therapy apparatus 6C includes the therapeutic X-ray head 10, the X-ray sources 97A and 97B and the sensor arrays 98A and 98B as the real time imager 30.

The X-ray head 10 is movably provided on the arc guide rail 9. The X-ray sources 97A and 97B are fixed on the sides of the X-ray head 10 that are different from each other in the Y-axis direction, respectively. The sensor arrays 98A and 98B are provided at the positions opposite to each other through the isocenter 5a in the X-ray sources 97A and 97B, respectively, while the position relation relative to the X-ray sources 97A and 97B is fixed. The X-ray sources 97A and 97B are provided on the sides opposite to each other, with the straight line between the X-ray head 10 and the isocenter 5a in FIG. 21.

This modification example is similar to the radio-therapy apparatus according to the embodiment in that the therapeutic X-ray head 10 is placed on the arc guide rail 9. Also, this modification example is similar to the second modification example in that the two sets of the X-ray sources 97A and 97B and the sensor arrays 98A and 98B are fixed to the X-ray head 10. Their relative positions are fixed in a predetermined range. The predetermined range is between 60 and 20 degrees, with regard to the angle between the sensor array 98A or the sensor array 98B, the isocenter 5a and the X-ray head 10. Preferably, it is between 45 and 30 degrees. This is determined in accordance with the condition that without any mutual influence between the X-ray head 10, the X-ray sources 97A and the X-ray source 97B, each of them is accurately operated and the diagnosis image having the sufficient precision is obtained.

Unlike the foregoing embodiment that includes the X-ray CT inspecting unit, the first modification example that the diagnostic X-ray source (CT X-ray tube) and the sensor array are provided on the rotating drum 99 and the second modification example that the two sets of the X-ray sources and the sensor arrays are provided on the rotating drum 99, the set of the X-ray source—the sensor array is connected to the X-ray head 10 and is operated to have a fixed position relation to the X-ray head 10 even under all of the radiation statuses.

Consequently, in addition to the obtainment of the effects of the operations in the foregoing respective embodiments, the set of the X-ray source and the sensor array has the fixed position relation to the X-ray head 10. Thus, it is possible to greatly reduce a burden on the control to obtain the diagnosis image and a burden on the operation of the real time imager. Also, since the sensor arrays 98A and 98B are provided on the side of the X-ray head 10, the therapeutic X-ray 3a which is very strong is never inputted to the sensor arrays 98A and 98B.

The X-ray head 10 is configured of the electric linear accelerator of 4 MeV to 10 MeV. As illustrated, the X-ray head 10 can be swung around the two shafts (the first and second swinging shafts S1, S2). That is, through these swinging operations, the non-isocentric radiation around the two shafts can be made possible, in addition to the isocentric radiation around the rotation shaft of the rotating drum.

As detailed above, according to the present invention, the radio-therapy apparatus can be provided, which can simplify the therapy plan after the radio-therapy is carried out on the specimen, and the method of operating the radio-therapy apparatus. Specifically, since the image of the diseased portion under the radio-therapy is recorded, it is possible to easily know the portion to which the therapeutic radiation is actually irradiated. Thus, it is sufficient as the record of the therapy history. Also, it is possible to easily establish the therapy plan after the execution of the radio-therapy.

Also, according to the present invention, the radio-therapy apparatus further includes the calculating unit for calculating the therapy history through the therapeutic radiation in accordance with the operation situation of the radiation head. The recording unit is configured to sequentially record the therapy history calculated by the calculating unit while relating it to the image of the diseased portion. Moreover, the calculating unit calculates the therapeutic dose amount to which the therapeutic radiation is applied and the estimated absorption dose amount estimated to be absorbed into the specimen, for each radiation direction of the therapeutic radiation, as the therapy history. Also, the radiation direction is determined from the radiation source coordinate indicating the radiation source of the radiation head and the target coordinate indicating the coordinate of the diseased portion of the specimen. Thus, the doctor can easily check the therapy situation, and determine whether or not the current therapy is proper, and plan the next therapy action.

Also, according to the present invention, the radio-therapy apparatus further includes the display for displaying the image of the diseased portion recorded on the recording unit and the therapy history. Thus, the doctor can quickly make the reasonable determination by viewing this display. Also, according to the present invention, the display further displays a transient value and an accumulation value of the radiation dose amounts of the therapeutic radiation. Thus, in addition to the data indicating the radiation states during the therapy, the portion to which the therapeutic radiation is irradiated and the dose amount at that time are displayed. Therefore, the doctor can easily check the therapy situation, plan the next therapy action and judge whether or not the present therapy is proper.

Also, according to the present invention, the display further displays the data indicating whether or not the therapy position is proper. Thus, it is possible to judge whether or not the therapy is proper, in accordance with the total data including even the situation of the therapy portion.

Moreover, according to the present invention, the recording unit further records the space coordinate of the diseased portion to which the therapeutic radiation from the radiation emitting head is irradiated. Thus, the radiation direction of the radiation becomes clear, which makes the collation with the therapy plan clear. Also, the doctor can easily check the therapy situation, judge whether or not the present therapy is proper and plan the next therapy action.

What is claimed is:

1. A radio-therapy apparatus comprising:
a radiation head configured to irradiate a therapeutic radiation;
an image processing section configured to generate an image of a diseased portion of a specimen from a result of detection of said diseased portion while tracking said diseased portion to which said therapeutic radiation is irradiated from said radiation head;
a control section configured to control said radiation head and said image processing section such that a period containing the generation of said image and the irradiation of said therapeutic radiation is repeated and the detection of said diseased portion in a next period is started prior to an end of a current period; and
a recording section configured to record said image of said diseased portion generated by said image processing section in order.

2. The radio-therapy apparatus according to claim 1, further comprising:
a calculating section configured to calculate a therapy record by said therapeutic radiation based on an operation state of said radiation head,
wherein said therapy record includes a therapeutic dose amount of said therapeutic radiation and an estimated absorption dose amount estimated to be absorbed by the specimen, and
said recording section records said therapy record calculated by said calculating section together with said image of said diseased portion in order.

3. The radio-therapy apparatus according to claim 2, wherein said calculating section calculates said therapy record every irradiation direction of said therapeutic radiation.

4. The radio-therapy apparatus according to claim 2, further comprising:
a display configured to display said image of said diseased portion and said therapy record which are recorded in said recording section.

5. The radio-therapy apparatus according to claim 4, wherein said display displays a moment value of said therapeutic radiation and an accumulation value of said therapeutic radiation.

6. The radio-therapy apparatus according to claim 5, wherein said display displays a data indicating whether a radiation field of said therapeutic radiation is proper.

7. The radio-therapy apparatus according to claim 1, wherein said recording section records a data indicating a position of said diseased portion to said radiation head in addition to said image.

8. The radio-therapy apparatus according to claim 1, wherein said control section controls said radiation head and said image processing section such that the irradiation of said therapeutic radiation in the current period is carried out after the detection of said diseased portion.

9. An operating method of a radio-therapy apparatus, comprising:
irradiating a therapeutic radiation;
generating an image of a diseased portion of a specimen from a result of detection of said diseased portion while tracking said diseased portion of a specimen to which therapeutic radiation is irradiated from a radiation head;
controlling said irradiating and said generating such that a period containing said generating said image and said irradiating said therapeutic radiation is repeated and the detection of said diseased portion in a next period is started prior to an end of a current period; and
recording said image of said diseased portion in order.

10. The operating method of the radio-therapy apparatus according to claim 9, further comprising:
calculating a therapy record by said therapeutic radiation based on an operation state of said radiation head,
wherein said therapy record includes a therapeutic dose amount of said therapeutic radiation and an estimated absorption dose amount estimated to be absorbed by the specimen, and
said recording comprises:
recording said therapy record together with said image of said diseased portion in order.

11. The operating method of the radio-therapy apparatus according to claim 10, wherein said calculating comprises:
calculating said therapy record every irradiation direction of said therapeutic radiation.

12. The operating method of the radio-therapy apparatus according to claim 10, further comprising:
displaying said image of said diseased portion and said therapy record.

13. The operating method of the radio-therapy apparatus according to claim 12, wherein said displaying comprises:
displaying a moment value of said irradiation dose amount of said therapeutic radiation and an accumulation value thereof.

14. The operating method of the radio-therapy apparatus according to claim 13, wherein said displaying comprises:
displaying a data indicating whether a radiation field of said therapeutic radiation is proper.

15. The operating method of the radio-therapy apparatus according to claim 9, wherein said recording comprises:
recording a data indicating a position of said diseased portion to said radiation head in addition to said image.

16. The operating method of the radio-therapy apparatus according to claim 9, wherein said controlling comprises:
controlling said irradiating and said generating such that the irradiation of said therapeutic radiation in the current period is carried out after the detection of said diseased portion.
